SEQUENCE LISTING (212 sequences)

SEQ ID NO: 1 - protein, 22 aa, Homo sapiens
IIYDRKFLMECRNSPVTKTPPR

SEQ ID NO: 2 - protein, 7 aa, Homo sapiens
KFLMECR

SEQ ID NO: 3 - protein, 10 aa, Homo sapiens
NSPVTKTPPR

SEQ ID NO: 4 - protein, 6 aa, Homo sapiens
FLMECR

SEQ ID NO: 5 - protein, 16 aa, Homo sapiens
DLKLENLMLDKDGHIK

SEQ ID NO: 6 - protein, 17 aa, Homo sapiens
EGWLHKRGEYIKTWRPR

SEQ ID NO: 7 - protein, 9 aa, Homo sapiens
ATGRYYAMK

SEQ ID NO: 8 - protein, 10 aa, Homo sapiens
LPFYNQDHEK

SEQ ID NO: 9 - protein, 17 aa, Homo sapiens
KLSPPFKPQVTSETDTR

SEQ ID NO: 10 - protein, 18 aa, Homo sapiens
KEVIVAKDEVAHTLTENR

SEQ ID NO: 11 - protein, 16 aa, Homo sapiens
HPFLTALKYSFQTHDR

SEQ ID NO: 12 - protein, 10 aa, Homo sapiens
ERVFSEDRAR

SEQ ID NO: 13 - protein, 7 aa, Homo sapiens
MYSQCVR

SEQ ID NO: 14 - protein, 7 aa, Homo sapiens
QLVHVVK

SEQ ID NO: 15 - protein, 17 aa, Homo sapiens
RFYQLTKLLDSVQPIAR

SEQ ID NO: 16 - protein, 20 aa, Homo sapiens
GAFQNLFQSVREVIQNPGPR

SEQ ID NO: 17 - protein, 6 aa, Homo sapiens
FFDELR

SEQ ID NO: 18 - protein, 22 aa, Homo sapiens
SFTNVNSRMLYFAPDLVFNEYR

SEQ ID NO: 19 - protein, 22 aa, Homo sapiens
SHMVSVDFPEMMAEIISVQVPK

SEQ ID NO: 20 - protein, 20 aa, Homo sapiens
SNPKSPQKPIVRVFLPNKQR

SEQ ID NO: 21 - protein, 7 aa, Homo sapiens
LLFQGFR

SEQ ID NO: 22 - protein, 16 aa, Homo sapiens
DLKSNNIFLHEDLTVK

SEQ ID NO: 23 - protein, 20 aa, Homo sapiens
DQIIFMVGRGYLSPDLSKVR

SEQ ID NO: 24 - protein, 12 aa, Homo sapiens
TFFTLAFCDFCR

SEQ ID NO: 25 - protein, 7 aa, Homo sapiens
LDALQQR

SEQ ID NO: 26 - protein, 11 aa, Homo sapiens
CGVTVRDSLKK

SEQ ID NO: 27 - protein, 11 aa, Homo sapiens
GLIPECCAVYR

SEQ ID NO: 28 - protein, 12 aa, Homo sapiens
QTAQGMDYLHAK

SEQ ID NO: 29 - protein, 9 aa, Homo sapiens
RLMAECLKK

SEQ ID NO: 30 - protein, 11 aa, Homo sapiens
SGTDVDAANLR

SEQ ID NO: 31 - protein, 8 aa, Homo sapiens
LFIIQACR

SEQ ID NO: 32 - protein, 8 aa, Homo sapiens
IFIIQACR

SEQ ID NO: 33 - protein, 9 aa, Homo sapiens
FSDLGFEVK

SEQ ID NO: 34 - protein, 11 aa, Homo sapiens
RGIALIFNHER

SEQ ID NO: 35 - protein, 20 aa, Homo sapiens
GNQHDVPVIPLDVVDNQTEK

SEQ ID NO: 36 - protein, 8 aa, Homo sapiens
EMFDPAEK

SEQ ID NO: 37 - protein, 18 aa, Homo sapiens
GHPAGGEENMTETDAFYK

SEQ ID NO: 38 - protein, 16 aa, Homo sapiens
VMLYQISEEVSRSELR

SEQ ID NO: 39 - protein, 8 aa, Homo sapiens
RVCAQINK

SEQ ID NO: 40 - protein, 22 aa, Homo sapiens
GDDILTILTEVNYEVSNKDDKK

SEQ ID NO: 41 - protein, 11 aa, Homo sapiens
QMPQPTFTLRK

SEQ ID NO: 42 - protein, 13 aa, Homo sapiens
TRTGSNIDCEKLR

SEQ ID NO: 43 - protein, 18 aa, Homo sapiens
IVNIFNGTSCPSLGGKPK

SEQ ID NO: 44 - protein, 10 aa, Homo sapiens
QMPGCFNFLR

SEQ ID NO: 45 - protein, 19 aa, Homo sapiens
LSKPTLENLTPVVLRPEIR

SEQ ID NO: 46 - protein, 9 aa, Homo sapiens
QLIIDLETR

SEQ ID NO: 47 - protein, 9 aa, Homo sapiens
LASYQAARK

SEQ ID NO: 48 - protein, 7 aa, Homo sapiens
VKLDSVR

SEQ ID NO: 49 - protein, 18 aa, Homo sapiens
SSDTEENVKRRTHNVLER

SEQ ID NO: 50 - protein, 14 aa, Homo sapiens
DQIPELENNEKAPK

SEQ ID NO: 51 - protein, 7 aa, Homo sapiens
HKLEQLR

SEQ ID NO: 52 - protein, 25 aa, Homo sapiens
KATAYILSVQAEEQKLISEEDLLRK

SEQ ID NO: 53 - protein, 15 aa, Homo sapiens
AMHVAQPAVVLASSR

SEQ ID NO: 54 - protein, 10 aa, Homo sapiens
AMDTGLYICK

SEQ ID NO: 55 - protein, 16 aa, Homo sapiens
EAGPPAFYRPNSDNRR

SEQ ID NO: 56 - protein, 19 aa, Homo sapiens
LASTNDKGSMAMESAKETR

SEQ ID NO: 57 - protein, 18 aa, Homo sapiens
QRDDGEGRGEVGSAGDMR

SEQ ID NO: 58 - protein, 11 aa, Homo sapiens
LLFAPNLLLDR

SEQ ID NO: 59 - protein, 9 aa, Homo sapiens
KCYEVGMMK

SEQ ID NO: 60 - protein, 23 aa, Homo sapiens
RSIQGNRHNDYMCPATNQCTIDK

SEQ ID NO: 61 - protein, 23 aa, Homo sapiens
SIQGHNDYMCPATNQCTIDKNRR

SEQ ID NO: 62 - protein, 19 aa, Homo sapiens
IADPEHDHTGFLTEYVATR

SEQ ID NO: 63 - protein, 10 aa, Homo sapiens
FRHENVIGIR

SEQ ID NO: 64 - protein, 7 aa, Homo sapiens
EIQILLR

SEQ ID NO: 65 - protein, 9 aa, Homo sapiens
NYLQSLPSK

SEQ ID NO: 66 - protein, 7 aa, Homo sapiens
ALDLLDR

SEQ ID NO: 67 - protein, 7 aa, Homo sapiens
TKVAWAK

SEQ ID NO: 68 - protein, 8 aa, Homo sapiens
ICDFGLAR

SEQ ID NO: 69 - protein, 8 aa, Homo sapiens
LFPKSDSK

SEQ ID NO: 70 - protein, 10 aa, Homo sapiens
NGKEFKPDHR

SEQ ID NO: 71 - protein, 15 aa, Homo sapiens
TSNRGHKVEVSWEQR

SEQ ID NO: 72 - protein, 14 aa, Homo sapiens
FKCPSSGTPNPTLR

SEQ ID NO: 73 - protein, 16 aa, Homo sapiens
GATPRDSGLYACTASR

SEQ ID NO: 74 - protein, 16 aa, Homo sapiens
HQHWSLVMESVVPSDR

SEQ ID NO: 75 - protein, 19 aa, Homo sapiens
VADPDHDHTGFLTEYVATR

SEQ ID NO: 76 - protein, 16 aa, Homo sapiens
DLKPSNLLLNTTCDLK

SEQ ID NO: 77 - protein, 8 aa, Homo sapiens
LFPNADSK

SEQ ID NO: 78 - protein, 9 aa, Homo sapiens
GQVFDVGPR

SEQ ID NO: 79 - protein, 9 aa, Homo sapiens
APEIMLNSK

SEQ ID NO: 80 - protein, 11 aa, Homo sapiens
LKELIFEETAR

SEQ ID NO: 81 - protein, 14 aa, Homo sapiens
ISELGAGNGGVVFK

SEQ ID NO: 82 - protein, 8 aa, Homo sapiens
IPEQILGK

SEQ ID NO: 83 - protein, 12 aa, Homo sapiens
DVKPSNILVNSR

SEQ ID NO: 84 - protein, 7 aa, Homo sapiens
SYMSPER

SEQ ID NO: 85 - protein, 9 aa, Homo sapiens
TLDQSPELR

SEQ ID NO: 86 - protein, 14 aa, Homo sapiens
WTLVNDETQAKMAR

SEQ ID NO: 87 - protein, 18 aa, Homo sapiens
LAMAGDTFTAEYVEFEVK

SEQ ID NO: 88 - protein, 16 aa, Homo sapiens
STAMDTLSSLVFQLGK

SEQ ID NO: 89 - protein, 11 aa, Homo sapiens
LMDTNTKGNKR

SEQ ID NO: 90 - protein, 11 aa, Homo sapiens
ELQHYVTMELR

SEQ ID NO: 91 - protein, 15 aa, Homo sapiens
HCADHFLNSEHKEIR

SEQ ID NO: 92 - protein, 7 aa, Homo sapiens
IVEDWQK

SEQ ID NO: 93 - protein, 9 aa, Homo sapiens
GNNLQDTLR

SEQ ID NO: 94 - protein, 19 aa, Homo sapiens
DFSHDDTLDVPTQVELLIK

SEQ ID NO: 95 - protein, 11 aa, Homo sapiens
QTTSPSGSLLR

SEQ ID NO: 96 - protein, 11 aa, Homo sapiens
APNTAELKICR

SEQ ID NO: 97 - protein, 17 aa, Homo sapiens
NSGSCLGGDEIFLLCDK

SEQ ID NO: 98 - protein, 8 aa, Homo sapiens
KRTYETFK

SEQ ID NO: 99 - protein, 14 aa, Homo sapiens
TPPYADPSLQAPVR

SEQ ID NO: 100 - protein, 13 aa, Homo sapiens
LPPVLSHPIFDNR

SEQ ID NO: 101 - protein, 16 aa, Homo sapiens
KSPFSGPTDPRPPPRR

SEQ ID NO: 102 - protein, 9 aa, Homo sapiens
KEIEAAIER

SEQ ID NO: 103 - protein, 14 aa, Homo sapiens
IQLGIDPYNAGSLK

SEQ ID NO: 104 - protein, 15 aa, Homo sapiens
EDISVVFSRASWEGR

SEQ ID NO: 105 - protein, 8 aa, Homo sapiens
LVQGSILK

SEQ ID NO: 106 - protein, 11 aa, Homo sapiens
CAGNEDIITLR

SEQ ID NO: 107 - protein, 7 aa, Homo sapiens
VSDYEMK

SEQ ID NO: 108 - protein, 15 aa, Homo sapiens
DLSHIGDAVVISCAK

SEQ ID NO: 109 - protein, 13 aa, Homo sapiens
FSASGELGNGNIK

SEQ ID NO: 110 - protein, 11 aa, Homo sapiens
SEGFDTYRCDR

SEQ ID NO: 111 - protein, 8 aa, Homo sapiens
MPSGEFAR

SEQ ID NO: 112 - protein, 23 aa, Homo sapiens
LFNVTSTLRINTTTNEIFYCTFR

SEQ ID NO: 113 - protein, 8 aa, Homo sapiens
LQDAGVYR

SEQ ID NO: 114 - protein, 9 aa, Homo sapiens
LFNVTSTLR

SEQ ID NO: 115 - protein, 7 aa, Homo sapiens
VNAPYNK

SEQ ID NO: 116 - protein, 11 aa, Homo sapiens
CMISYGGADYK

SEQ ID NO: 117 - protein, 12 aa, Homo sapiens
LNTEETVKVHVR

SEQ ID NO: 118 - protein, 13 aa, Homo sapiens
ALETSVAADFYHR

SEQ ID NO: 119 - protein, 16 aa, Homo sapiens
DHESVFTVSLWDCDRK

SEQ ID NO: 120 - protein, 11 aa, Homo sapiens
FEPYHDSALAR

SEQ ID NO: 121 - protein, 9 aa, Homo sapiens
SFLGINKER

SEQ ID NO: 122 - protein, 11 aa, Homo sapiens
YQVVQTLDCLR

SEQ ID NO: 123 - protein, 10 aa, Homo sapiens
MAEVASRDPK

SEQ ID NO: 124 - protein, 15 aa, Homo sapiens
KTSPHFQKFQDICVK

SEQ ID NO: 125 - protein, 16 aa, Homo sapiens
TQDQQSLSDVEGAYSR

SEQ ID NO: 126 - protein, 13 aa, Homo sapiens
KCCQAGMVLGGRK

SEQ ID NO: 127 - protein, 16 aa, Homo sapiens
FYQLTKLLDNLHDLVK

SEQ ID NO: 128 - protein, 20 aa, Homo sapiens
ALSVEFPEMMSEVIAAQLPK

SEQ ID NO: 129 - protein, 9 aa, Homo sapiens
SSYIRELIK

SEQ ID NO: 130 - protein, 24 aa, Homo sapiens
RAMEGQHNYLCAGRNDCIVDKIRR

SEQ ID NO: 131 - protein, 23 aa, Homo sapiens
ALDAVALPQPVGVPNESQALSQR

SEQ ID NO: 132 - protein, 22 aa, Homo sapiens
SYKHVSGQMLYFAPDLILNEQR

SEQ ID NO: 133 - protein, 18 aa, Homo sapiens
IYNLCAERHYDTAKFNCR

SEQ ID NO: 134 - protein, 12 aa, Homo sapiens
AQEALDFYGEVR

SEQ ID NO: 135 - protein, 12 aa, Homo sapiens
DKKGVTIPSQRR

SEQ ID NO: 136 - protein, 12 aa, Homo sapiens
VKIYSSNSGPTR

SEQ ID NO: 137 - protein, 7 aa, Homo sapiens
YFSPNFK

SEQ ID NO: 138 - protein, 8 aa, Homo sapiens
NNIDDVVR

SEQ ID NO: 139 - protein, 19 aa, Homo sapiens
ADNDKEYLVLTLTKNDLDK

SEQ ID NO: 140 - protein, 12 aa, Homo sapiens
ISAFGYLECSAK

SEQ ID NO: 141 - protein, 16 aa, Homo sapiens
FKRFPCLSLLSSWGYR

SEQ ID NO: 142 - protein, 8 aa, Homo sapiens
EVFEMATR

SEQ ID NO: 143 - protein, 15 aa, Homo sapiens
HFCPNVPIILVGNKK

SEQ ID NO: 144 - protein, 13 aa, Homo sapiens
KKLVIVGDGACGK

SEQ ID NO: 145 - protein, 12 aa, Homo sapiens
IGAFGYMECSAK

SEQ ID NO: 146 - protein, 17 aa, Homo sapiens
QVELALWDTAGQEDYDR

SEQ ID NO: 147 - protein, 18 aa, Homo sapiens
DGVREVFEMATRAALQAR

SEQ ID NO: 148 - protein, 18 aa, Homo sapiens
LGAGPGDAGEVQAHPFFR

SEQ ID NO: 149 - protein, 16 aa, Homo sapiens
FSLSGGYWNSVSDTAK

SEQ ID NO: 150 - protein, 8 aa, Homo sapiens
LTAALVLR

SEQ ID NO: 151 - protein, 17 aa, Homo sapiens
HPWIVHWDQLPQYQLNR

SEQ ID NO: 152 - protein, 16 aa, Homo sapiens
DSPGIPPSANAHQLFR

SEQ ID NO: 153 - protein, 8 aa, Homo sapiens
TSFTSVSR

SEQ ID NO: 154 - protein, 10 aa, Homo sapiens
YEELQQTAGR

SEQ ID NO: 155 - protein, 9 aa, Homo sapiens
AQYEEIANR

SEQ ID NO: 156 - protein, 9 aa, Homo sapiens
EYQELMNTK

SEQ ID NO: 157 - protein, 9 aa, Homo sapiens
FVSTTSSSR

SEQ ID NO: 158 - protein, 9 aa, Homo sapiens
EYQELMNVK

SEQ ID NO: 159 - protein, 11 aa, Homo sapiens
TAAENEFVTLK

SEQ ID NO: 160 - protein, 9 aa, Homo sapiens
EELQVTAGR

SEQ ID NO: 161 - protein, 10 aa, Homo sapiens
SGFSSISVSR

SEQ ID NO: 162 - protein, 17 aa, Homo sapiens
ATGGGLSSVGGGSSTIK

SEQ ID NO: 163 - protein, 9 aa, Homo sapiens
LDADPSLQR

SEQ ID NO: 164 - protein, 12 aa, Homo sapiens
GQLEALQVDGGR

SEQ ID NO: 165 - protein, 9 aa, Homo sapiens
DVDAAYMSK

SEQ ID NO: 166 - protein, 8 aa, Homo sapiens
NEISEMNR

SEQ ID NO: 167 - protein, 8 aa, Homo sapiens
LLEGEESR

SEQ ID NO: 168 - protein, 9 aa, Homo sapiens
QWYETNAPR

SEQ ID NO: 169 - protein, 9 aa, Homo sapiens
LEQEIATYR

SEQ ID NO: 170 - protein, 12 aa, Homo sapiens
TTEYQLSTLEER

SEQ ID NO: 171 - protein, 10 aa, Homo sapiens
TVVQEVVDGK

SEQ ID NO: 172 - protein, 15 aa, Homo sapiens
VLQIDNAKLAAEDFR

SEQ ID NO: 173 - protein, 8 aa, Homo sapiens
DLGSELVR

SEQ ID NO: 174 - protein, 17 aa, Homo sapiens
SVSPTTEMVSNESVDYR

SEQ ID NO: 175 - protein, 17 aa, Homo sapiens
SVSPTTEMVSNESVDYR

SEQ ID NO: 176 - protein, 6 aa, Homo sapiens
NMLDEK

SEQ ID NO: 177 - protein, 6 aa, Homo sapiens
NMVDEK

SEQ ID NO: 178 - protein, 13 aa, Homo sapiens
DMYDKEYYSVHNK

SEQ ID NO: 179 - protein, 13 aa, Homo sapiens
DMHDKEYYSVHNK

SEQ ID NO: 180 - protein, 13 aa, Homo sapiens
DMYDKEYYSVHNK

SEQ ID NO: 181 - protein, 13 aa, Homo sapiens
DMYDKEYYSVHNK

SEQ ID NO: 182 - protein, 13 aa, Homo sapiens
DMYDKEYYSVHNK

SEQ ID NO: 183 - protein, 11 aa, Homo sapiens
WMALESLQTQK

SEQ ID NO: 184 - protein, 11 aa, Homo sapiens
WTALESLQTQK

SEQ ID NO: 185 - protein, 8 aa, Homo sapiens
YSFGATVK

SEQ ID NO: 186 - protein, 10 aa, Homo sapiens
VNGIGIGEFK

SEQ ID NO: 187 - protein, 16 aa, Homo sapiens
NTSISGDLHILPVAFR

SEQ ID NO: 188 - protein, 8 aa, Homo sapiens
DPPFCVAR

SEQ ID NO: 189 - protein, 9 aa, Homo sapiens
GMSYLEDVR

SEQ ID NO: 190 - protein, 8 aa, Homo sapiens
ELVSEFSR

SEQ ID NO: 191 - protein, 19 aa, Homo sapiens
SGGGDLTLGLEPSEEEAPR

SEQ ID NO: 192 - protein, 19 aa, Homo sapiens
SGGGDLTLGLEPSEEEAPR

SEQ ID NO: 193 - protein, 19 aa, Homo sapiens
SGGGDLTLGLEPSEEEAPR

SEQ ID NO: 194 - protein, 19 aa, Homo sapiens
SGGGDLTLGLEPSEEEAPR

SEQ ID NO: 195 - protein, 15 aa, Homo sapiens
GLQSLPTHDPSPLQR

SEQ ID NO: 196 - protein, 15 aa, Homo sapiens
GLQSLPTHDPSPLQR

SEQ ID NO: 197 - protein, 15 aa, Homo sapiens
GLQSLPTHDPSPLQR

SEQ ID NO: 198 - protein, 15 aa, Homo sapiens
GLQSLPTHDPSPLQR

SEQ ID NO: 199 - protein, 11 aa, Homo sapiens
LVVVGAGGVGK

SEQ ID NO: 200 - protein, 15 aa, Homo sapiens
VKDSEDVPMVLVGNK

SEQ ID NO: 201 - protein, 13 aa, Homo sapiens
DSEDVPMVLVGNK

SEQ ID NO: 202 - protein, 12 aa, Homo sapiens
SYGIPFIETSAK

SEQ ID NO: 203 - protein, 12 aa, Homo sapiens
QGVDDAFYTLVR

SEQ ID NO: 204 - protein, 18 aa, Homo sapiens
FAIQYGTGRVDGILSEDK

SEQ ID NO: 205 - protein, 9 aa, Homo sapiens
VDGILSEDK

SEQ ID NO: 206 - protein, 9 aa, Homo sapiens
FAIQYGTGR

SEQ ID NO: 207 - protein, 9 aa, Homo sapiens
VGPGLTLAK

SEQ ID NO: 208 - protein, 11 aa, Homo sapiens
SATWTYSTELK

SEQ ID NO: 209 - protein, 15 aa, Homo sapiens
EFNEGQIAPPSHLIR

SEQ ID NO: 210 - protein, 6 aa, Homo sapiens
ICAPGR

SEQ ID NO: 211 - protein, 8 aa, Homo sapiens
ETYEMLLK

SEQ ID NO: 212 - protein, 16 aa, Homo sapiens
TPSSASTVSVGSSETR

(19) United States
(12) Patent Application Publication
(10) Pub. No.: US 2018/0129779 A1
(43) Pub. Date: May 10, 2018

(54) PROTEOGENOMIC ANALYSIS SYSTEM AND METHODS

(71) Applicant: RICHARD-ALLAN SCIENTIFIC COMPANY, Kalamazoo, MI (US)

(72) Inventors: Jared Isaac, Portage, MI (US); Amol Prakash, Shrewsbury, MA (US); Jennifer Freeland, Portage, MI (US)

(21) Appl. No.: 15/666,174

(22) Filed: Aug. 1, 2017

Related U.S. Application Data
(63) Continuation-in-part of application No. 15/347,706, filed on Nov. 9, 2016.

(51) Int. Cl. G06F 19/18; G01N 33/68; C12Q 1/68; G06T 7/00; G06T 11/60

(52) U.S. Cl. CPC G06F 19/18; G01N 33/68; C12Q 1/68; G06T 7/0012; G06T 11/001; G06T 2207/30024; G06T 2207/30072; G06T 2207/20221; G06T 11/60

(57) ABSTRACT

Embodiments are directed to methods for representing cell content of a tissue sample. One method includes identifying a pixel in a digital image of a tissue sample. The identified pixel is mapped to a portion of the tissue sample. A first data set is accessed representing a genomics analysis of nucleic acids that are separated from tissue proteins in the tissue sample. A second data set is also accessed representing a proteomic analysis of proteins that were separated from the nucleic acids of the tissue sample. The data sets are mapped to a renderable attribute, so that the renderable attribute has a specified value based on the mapped data sets. Then, a portion of a representation is rendered for the identified pixel, according to the specified value of the renderable attribute. The representation includes an indication of how the separated nucleic acids and proteins are distributed within the tissue sample.

### CROSS-REFERENCE TO RELATED APPLICATIONS

This application is a continuation-in-part which claims the benefit of and priority to U.S. patent application Ser. No. 15/347,706, entitled “PROTEOGENOMIC ANALYSIS SYSTEM AND METHODS,” filed on Nov. 9, 2016, which application is incorporated by reference herein in its entirety.

### BACKGROUND

### 1. Technical Field

The present disclosure relates to isolation of nucleic acid and protein molecules from a biological sample and, more specifically, to systems, methods, and products for isolating proteogenomic material from a single section of formalin-fixed, paraffin-embedded (FFPE) tissue.

### 2. Relevant Technology

Formalin-fixed, paraffin-embedded (FFPE) tissue is a common method for clinical sample preservation and archiving. FFPE tissue samples can be sectioned into thin slices of the tissue with a microtome or cryostat and analyzed for pathological, histological, and molecular biological characteristics to diagnose disease and other tissue conditions.

Historically, FFPE samples were not considered to be a viable source for molecular analyses. Recently, however, it has been discovered that with appropriate processing, a sufficient amount of DNA or RNA can be isolated from FFPE samples. The purified nucleic acids may even be suitable for downstream genomic and gene expression analyses, such as polymerase chain reaction (PCR), quantitative reverse transcription PCR (qRT-PCR), microarray, array comparative genomic hybridization (CGH), microRNA, next-generation sequencing (NGS), and methylation profiling. FFPE samples can alternatively be processed to isolate proteins or peptides suitable for downstream proteomic analysis, including mass spectrometry (MS) or immunoassay.

FFPE processing techniques and reagents suitable for isolation of certain cellular material are not known to be suitable for isolation of other cellular material. For example, harsh detergents and other reaction conditions (such as time and temperature) used in processing FFPE samples for the isolation of nuclear DNA are not conducive to isolating proteins or RNA suitable for analysis. Similarly, using mild reagents or reaction conditions optimal for protein isolation and analysis are not known to be robust enough for purification of nuclear DNA and may be destructive to RNA. Likewise, conditions for isolating RNA for further analysis are not suitable for isolation and analysis of DNA and protein.

To avoid these and other problems, separate FFPE sections have been processed for isolation and analysis of DNA, RNA, and proteins, respectively. A major drawback to using separate sections is the risk of obtaining misleading or conflicting genomic and proteomic data. For instance, in some cases, even adjacent or sequential sections contain cells having different genomic and proteomic profiles. Moreover, biopsied tissue samples are often small, such that a limited number of microtome or cryostat sections are available. Using separate sections for each assay may diminish the supply of the tissue sample available for follow-up studies.

Accordingly, systems, methods, and products that address some or all of the above shortcomings and other deficiencies known in the art are needed.

### BRIEF SUMMARY

Embodiments of the present disclosure solve one or more of the foregoing or other problems in the art with systems, methods, and products for isolating nucleic acid and protein molecules from a formalin-fixed, paraffin-embedded (FFPE) tissue sample. An illustrative embodiment includes extracting DNA, RNA and proteins from a single thin section of FFPE tissue sample. The method can include providing a biological sample that has a plurality of cells that contain nucleic acids (e.g., DNA and/or RNA) and proteins. The method can include preparing a lysate of the cells such that the lysate contains the nucleic acids and proteins under conditions that permit extraction of nucleic acids and proteins that are suitable for molecular biological analysis. For instance, in some embodiments, the biological sample (e.g., tissue section) can be incubated in a lysis buffer. The buffer conditions, reaction time, and/or temperature of the lysis reaction can be adapted or configured such that a suitable amount of nucleic acid and protein are released and in stable condition for separation and proteogenomic analysis.

In some embodiments, the method can include (sequentially) alkylating, reducing, diluting, and/or enzymatically digesting proteins in the lysate. Suitable amounts and/or types of alkylating agent, reducing agent, diluting agent, and/or protease can maintain the suitability of the proteins (or peptides) for proteomic analysis. Nucleic acids can be separated from (digested) proteins (or peptides) present in the lysate or reaction sample. Nucleic acids can be quantified (e.g, by fluorimeter (or fluorometer), spectrophotometer, bioanalyzer, etc.), amplified (e.g., by PCR), and/or sequenced (e.g., by NGS) in a variety of ways and through a variety of means. Mass spectroscopic analysis (e.g., liquid chromatography-mass spectrometry (LC-MS)) of the separated digested proteins can also be performed.

Systems and products for performing methods can include reagents and apparatus for performing steps of the foregoing or other methods described herein. Panels for detecting the presence and level of expression of peptides to differentiate between disease states (e.g., cancer subtypes) are also contemplated and described herein. Such panels can include a plurality of peptides adapted or configured to detect and/or quantify specific proteins or peptides present in the sample.

In further embodiments, a method is provided for representing cell content of a tissue sample. The method includes identifying a pixel in a digital image of a tissue sample. The identified pixel is mapped to a portion of the tissue sample. Next, the method includes accessing a first data set representing a genomics analysis of nucleic acids that are separated from tissue proteins in the tissue sample, and accessing a second data set representing a proteomic analysis of proteins that were separated from the nucleic acids of the tissue sample. The method further includes mapping the accessed first and second data sets to a renderable attribute, so that the renderable attribute has a specified value based on the mapped data sets, and rendering at least a portion of a representation corresponding to the identified pixel, according to the specified value of the renderable attribute. The representation includes an indication of how the separated nucleic acids and proteins are distributed within the tissue sample.

Additional features and advantages of exemplary embodiments of the present disclosure will be set forth in the description which follows, and in part will be obvious from the description, or may be learned by the practice of such exemplary embodiments. The features and advantages of such embodiments may be realized and obtained by means of the instruments and combinations particularly pointed out in the appended claims. These and other features will become more fully apparent from the following description and appended claims, or may be learned by the practice of such exemplary embodiments as set forth hereinafter.

It is also noted that each of the foregoing, following, and/or other features described herein can represent a distinct embodiment of the present disclosure. Moreover, combinations of any two or more of such features represent distinct embodiments of the present disclosure. Such embodiments can also be combined in any suitable combination and/or order without departing from the scope of this disclosure. Thus, each of the features described herein can be combinable with any one or more other features described herein in any suitable combination and/or order. Accordingly, the present disclosure is not limited to the specific combinations of exemplary embodiments described in detail herein.

### BRIEF DESCRIPTION OF THE DRAWINGS

In order to describe the manner in which certain advantages and features of the present disclosure can be obtained, a description of the disclosure will be rendered by reference to specific embodiments thereof which are illustrated in the appended drawings. Understanding that these drawings depict only typical embodiments of the disclosure and are not therefore to be considered to be limiting of its scope, the disclosure will be described and explained with additional specificity and detail through the use of the accompanying drawings in which:

FIG. 1 is a flowchart depicting a protocol for the isolation of proteogenomic material from a biological sample in accordance with an embodiment of the present disclosure.

FIG. 2 illustrates a computer architecture in which embodiments described herein may operate including representing cell content of a tissue sample.

FIG. 3 illustrates a flowchart of an example method for representing cell content of a tissue sample.

FIG. 4 illustrates an example digital image of a tissue sample.

FIG. 5 illustrates an example differential image.

FIG. 6 illustrates an example image of a heat map overlay.

FIG. 7 illustrates an alternative example image of a heat map overlay.

### DETAILED DESCRIPTION

Before describing various embodiments of the present disclosure in detail, it is to be understood that this disclosure is not limited to the specific parameters and description of the particularly exemplified systems, methods, and/or products that may vary from one embodiment to the next. Thus, while certain embodiments of the present disclosure will be described in detail, with reference to specific configurations, parameters, components, reagents, etc., the descriptions are illustrative and are not to be construed as limiting the scope of the present disclosure and/or the claimed invention. In addition, the terminology used herein is for the purpose of describing the embodiments, and is not necessarily intended to limit the scope of the present disclosure and/or the claimed invention.

Unless defined otherwise, all technical and scientific terms used herein have the same meaning as commonly understood by one of ordinary skill in the art to which the present disclosure pertains.

Various aspects of the present disclosure, including systems, methods, and/or products may be illustrated with reference to one or more embodiments or implementations, which are exemplary in nature. As used herein, the terms “embodiment” and implementation” mean “serving as an example, instance, or illustration,” and should not necessarily be construed as preferred or advantageous over other aspects disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the description thereof.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

As used in this specification and the appended claims, the singular forms “a,” “an” and “the” also contemplate plural referents, unless the context clearly dictates otherwise. Thus, for example, reference to a “nucleic acid” includes one, two, or more nucleic acid or types of nucleic acid. Similarly, reference to a plurality of referents should be interpreted as comprising a single referent and/or a plurality of referents unless the content and/or context clearly dictate otherwise. Thus, reference to “nucleic acids” does not necessarily require a plurality of such nucleic acids or a plurality of types of nucleic acids. Instead, it will be appreciated that independent of conjugation; one or more nucleic acids or types thereof are contemplated herein.

It will also be appreciated that where two or more values, or a range of values (e.g., less than, greater than, at least, and/or up to a certain value, and/or between two recited values) is disclosed or recited, any specific value or range of values falling within the disclosed values or range of values is likewise disclosed and contemplated herein. Thus, disclosure of an illustrative measurement (e.g., volume, concentration, etc.) that is less than or equal to about 10 units or between 0 and 10 units includes, illustratively, a specific disclosure of: (i) a measurement of 9 units, 5 units, 1 units, or any other value between 0 and 10 units, including 0 units and/or 10 units; and/or (ii) a measurement between 9 units and 1 units, between 8 units and 2 units, between 6 units and 4 units, and/or any other range of values between 0 and 10 units.

In certain embodiments, the ordering and/or positioning of certain method steps and/or system components can contribute to and even determine the effectiveness and/or functionality of the embodiment. In addition, performance of a first step before a second step can provide useful pre-processing and can alter the outcome of the second step. Likewise, performance of a second step after a first step can be useful in determining the outcome of the second step.

To facilitate understanding, like references (i.e., like naming and/or numbering of components and/or elements) have been used, where possible, to designate like components and/or elements common to the written description and/or figures. Nevertheless it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

The headings used herein are for organizational purposes only and are not meant to be used to limit the scope of the description or the claims.

The present disclosure relates to systems, methods, and products for isolating nucleic acid and protein molecules from a biological sample, such as a single formalin-fixed, paraffin-embedded (FFPE) tissue sample section. Certain methods can include: (i) providing a biological sample that has a plurality of cells that contain nucleic acids (e.g., DNA and/or RNA) and proteins, (ii) preparing a lysate of the cells under conditions that permit extraction of nucleic acids and proteins that are suitable for molecular biological analysis such that the lysate contains the nucleic acids and proteins, (iii) alkylating, reducing, diluting, and/or enzymatically digesting proteins in the lysate, (iv) separating nucleic acids present in the lysate or reaction sample from digested proteins or peptides present in the lysate or reaction sample, and/or (v) performing molecular biological analysis, such as next generation sequencing (NGS) and/or mass spectroscopy of the separated nucleic acids and/or proteins or peptides.

Methods can enable users to isolate RNA, DNA, and protein from the same section, piece, and/or FFPE tissue further enabling users to correlate RNA, DNA, and protein status and/or characteristics from the same portion of a tissue. The risk of obtaining misleading or conflicting genomic and proteomic data can thereby be decreased because proteogenomic material from the same section and/or same cells are involved in the analysis. Further, because a single thin section (approximately 7 micron in thickness) can be used for both nucleic acid and protein analytics, the remainder of the FFPE tissue block can be available for further analysis as may be needed for later studies.

Systems and products for performing methods can include reagents and apparatus for performing steps of the foregoing or other methods described herein. For instance, two or more apparatus can be coupled together or arranged in fluid communication so as to form a system. In addition, peptide panels for detecting the presence and level of expression of peptides to differentiate between disease states (e.g., cancer subtypes) can include a plurality of peptides adapted or configured to detect and/or quantify specific proteins or peptides present in the sample.

As used herein, the term “systems” also contemplates devices, apparatus, compositions, assemblies, kits, and so forth. Similarly, the term “method” also contemplates processes, procedures, steps, and so forth. Moreover, the term “products” also contemplates devices, apparatus, compositions, assemblies, kits, and so forth.

In at least one embodiment, the terms “form,” “forming,” and the like are open-ended, such that components that are combined, mixed, coupled, etc. so as to form a system, assembly, mixture, etc. do not necessarily constitute the entire system, assembly, mixture, etc. Accordingly, the system, assembly, mixture, etc. can comprise said components, without, necessarily, consisting, either entirely or essentially, of said components.

As used herein, the terms “mixture,” “fluid mixture,” “liquid mixture,” and the like can comprise any suitable composition and/or combination of the specific components thereof. For instance, a fluid or liquid mixture can comprise a solution, suspension, colloid, emulsion, or other mixture of liquid and/or non-liquid components.

As used herein, the term “biological” refers to organisms (e.g., microbes, such as bacteria, yeast, etc., plants, animals, etc.), whether living or non-living, and/or components thereof or produced thereby, including cells, molecules/compounds (e.g., nucleic acids, proteins, fats, fatty acids, etc.), or combination(s), aggregate(s), crystal(s), or precipitate(s) thereof.

As used herein, the terms “coupled”, “attached”, “connected,” and/or “joined” are used to indicate either a direct association between two components or, where appropriate, an indirect association with one another through intervening or intermediate components. In contrast, when a component is referred to as being “directly coupled”, “directly attached”, “directly connected,” and/or “directly joined” to another component, no intervening elements are present or contemplated.

Furthermore, aspects of the present disclosure can be illustrated by describing components that are in fluid communication or fluidly coupled, connected, etc. Such fluid communication or connection will be understood by those skilled in the art to imply at least one route or flow path between the components. Generally, such fluid communication or connection involves at least one fluid inlet and/or fluid outlet disposed between components in fluid communication and/or for effectuating the fluid connection. In addition, “fluid connections,” “fluid couplings,” and the like, as used herein, can comprise fluid flow paths, such as those found within fluid lines, tubes, etc.

Reference will now be made to the figures of the present disclosure. It is noted that the figures are not necessarily drawn to scale and that the size, order, orientation, position, and/or relationship of or between various components illustrated in the figures can be altered in some embodiments without departing from the scope of this disclosure.

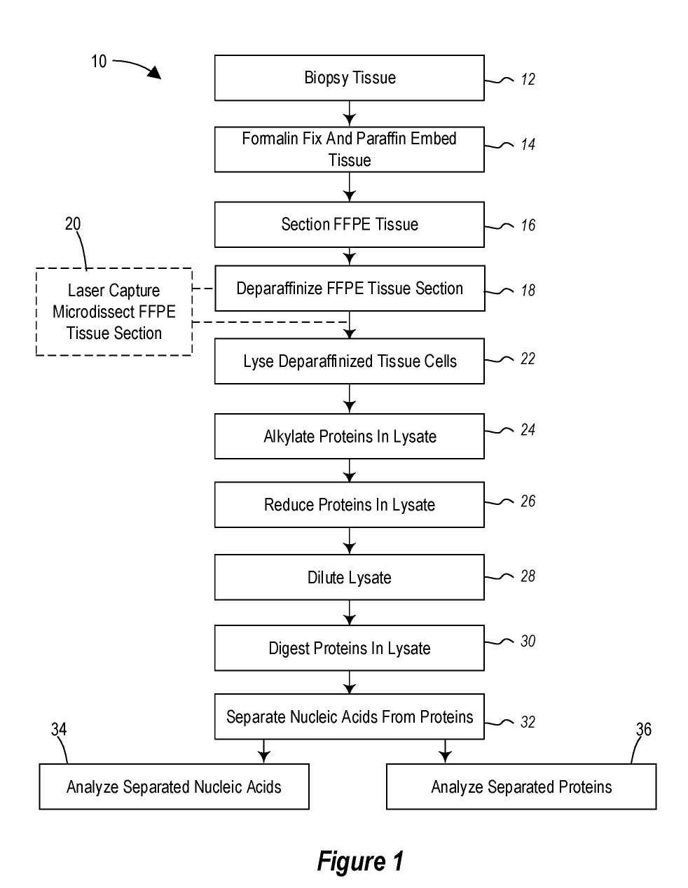

FIG. 1 is a flowchart depicting a protocol or method 10 for the isolation of proteogenomic material from a biological sample (e.g., a single section of FFPE tissue sample). It will be appreciated that FIG. 1 illustrates various steps that can be useful in practicing certain aspects of the present disclosure. Embodiments of the present disclosure can, however, include fewer steps and/or additional steps than those explicitly illustrated in FIG. 1.

Illustratively, an embodiment can include a step 12 of performing a tissue biopsy and/or providing biopsy tissue. Step 12 can be performed, for example by a surgeon. The tissue can be or comprise any suitable biological tissue type, whether diseased or healthy, cancerous (malignant) or benign, necrotic or living. In at least one embodiment, the tissue can be or comprise cancerous tissue, such as a tumor or other mass. Accordingly, the biopsy tissue can comprise a tumor or other biopsy in certain embodiments. A list of cancers that can be biopsied or otherwise sampled to provide tissue useful in embodiments of the present disclosure can be found at cancer.gov/types, the list being incorporated herein by specific reference.

In at least one embodiment, the tissue can comprise small cell or non-small cell lung cancer or tumor tissue. In some embodiments, the tissue can comprise one or more subtypes of lung cancer, such as squamous cell (epidermoid) carcinoma, adenocarcinoma, adenosquamous carcinoma, sarcomatoid carcinoma, and so forth. Certain embodiments of the present disclosure can be useful in distinguishing cancer subtypes. In some embodiments, the tissue can comprise breast cancer or tumor tissue. It will be appreciated that other cancer types and/or subtypes are also contemplated herein.

Some embodiments can include a step 14 of formalin fixing and paraffin embedding the tissue sample. Systems, methods, and products for formalin fixing and paraffin embedding tissue are known in the art and contemplated herein. It will also be appreciated that some embodiments can include using fresh or fresh-frozen tissue. The tissue can then be sectioned or otherwise prepared for processing. For instance, certain embodiments can include a step 16 of sectioning FFPE tissue. A thin section of FFPE or fresh-frozen tissue block can be made (e.g., cut) using a microtome or cryostat instrument, such as those commercially available from Thermo Fisher Scientific. In some embodiments, FFPE tissue sections (or slices) can be between 50 nanometers (nm) and 100 micrometers or micron (μm) in thickness, preferably between about 3-20 μm, more preferably between about 5-10 μm, most preferably about 7 am.

Some embodiments can include a step 18 of deparaffinizing the FFPE tissue section, as known in the art. For instance, a single FFPE tissue section can be transferred to and/or disposed in a container, such as a sample tube, sample well, or receptacle, which can have a volume of between about 0.5-15 milliliters (mL), preferably between about 1-5 mL, more preferably between about 1.5-2.5 mL, most preferably about 2 mL, in certain embodiments.

In certain embodiments, the FFPE tissue section can be mixed with an organic clearant, such as xylene, which can be applied to the tissue section and/or added to the container. The sample can be collected from the mixture, for example, via centrifugation at room temperature (RT) or other temperature.

The sample and/or tubes can be heated for 1-10 minutes, preferably for about 3 minutes, at between about 20-100° C., preferably between about 37-65° C., more preferably between about 42-58° C., most preferably about 56° C., to melt paraffin. Heated samples can be centrifuged (at RT or other temperature), at between about 1-20,000 rpm, preferably between about 1,000-15,000 rpm, more preferably between about 5,000-12,000 rpm, most preferably about 12,000 rpm and/or for between about 1-10 minutes, preferably between about 2-5 minutes, more preferably about 2 minutes, to pellet the tissue.

Xylene can be removed from the container and/or pelleted tissue without disturbing pellet by decanting, pipetting, etc. The pellet can then be mixed with an organic solvent, such as methanol (MeOH), ethanol (EtOH), or isopropanol, preferably EtOH. For instance, between 0.5-2 mL, preferably 1 mL of 10-100% (in water), preferably 100% EtOH can be added to the pellet. The sample can be centrifuged (at RT or other temperature) at between about 1-20,000 rpm, preferably between about 1,000-15,000 rpm, more preferably between about 5,000-12,000 rpm, most preferably about 12,000 rpm and/or for between about 1-10 minutes, preferably between about 2-5 minutes, more preferably about 2 minutes to pellet the tissue.

The organic solvent can be removed from the container and/or pelleted tissue without disturbing pellet, by decanting, pipetting, etc. The pellet can be mixed one or more additional times, successively, with an organic solvent as described above. The pellet can be dried, such as by vacuum, air flow, or passively (for between about 1-20 minutes, preferably about 15 minutes, at between about 20-100° C., preferably about 37° C.) until the pellet is dry and/or essentially all solvent is removed. The pellet, comprising the deparaffinized tissue sample, can then be used to prepare the multi-analyte lysate as described further herein.

In at least one embodiment, the tissue section can be deparaffinized and/or selected areas of the FFPE tissue section can be isolated, such as by laser capture microdissection (LCM), as in step 20. For instance, FFPE tissue sections can be adhered to glass or an LCM specialty slide, such as a polyethylene naphthalate (PEN) membrane slide. For instance, the slide and/or adhered tissue section can be treated one or more times (e.g., 2, 3, 4, or 5 times), successively, with and/or in a suitable amount of an organic clearant, such as xylene. Each dewaxing treatment can be for 1-5 minutes, preferably 3 minutes.

The slide and/or adhered tissue section can then be treated one or more times (e.g., 2, 3, 4, or 5 times), successively, with a suitable amount of an organic solvent, such as MeOH, EtOH, or isopropanol, preferably 10-100% EtOH, more preferably 100% EtOH. The tissue can then be stained, such as with heamatoxylin and/or eosin and/or, preferably, the Arcturus® Paradise® Plus stain product available commercially from Thermo Fisher Scientific. The staining step can be for between about 0.1-10 minutes, preferably between about 0.5-1 minutes. The stained sample can be dried (or dehydrated), such as through graded and/or successive EtOH/xylene treatments. The slides can (then) be stored (e.g., at 4° C.) until LCM is performed, for example using an ArcturusXT™ LCM instrument available commercially from Thermo Fisher Scientific. Samples dissected from a tissue section can be captured in LCM caps and/or can be used to prepare multi-analyte lysate as described further herein.

An illustrative slide-adhered tissue section processing protocol is outlined below:

Xylene—3 min

Xylene—3 min

Xylene—3 min

Xylene—3 min

100% Ethanol—1 min

100% Ethanol—1 min

95% Ethanol—1 min

H₂O—1 min

Stain—0.5 min. (7 μm) and 1 min (20 μm)

H₂O—1 min

100% Ethanol—1 min

100% Ethanol—1 min

100% Ethanol—1 min

Xylene—3 min

Xylene—3 min

Xylene—3 min

In at least one embodiment, a whole section of tissue can be used for global correlation of proteogenomic data. In at least one embodiment, laser capture microdissection can be used for targeted selection of specific cell types.

Some embodiments can include preparing a multi-analyte lysate. For instance, an embodiment can include a step 22 of lysing the deparaffinized FFPE sample. Cells of the deparaffinized FFPE tissue sample section can be lysed, such as by heat lysis in a suitable lysis buffer, for a suitable period of time. In particular, cell lysis can be performed under conditions that permit extraction of nucleic acids (e.g., DNA and/or RNA) and proteins that are suitable or in a condition for genomic and proteomic analysis. For example, the buffer conditions, reaction time, and temperature of the lysis reaction can be adapted or configured such that a suitable amount of DNA, RNA, and proteins are released and in stable condition for separation and proteogenomic analysis.

In at least one embodiment, the lysis buffer (or solution) can include a denaturing agent, such as guanidine HCl, at a concentration between about 0-8M, a buffering agent, such as Tris(hydroxymethyl)aminomethane hydrochloride (Tris-HCl), at a concentration between about 0-250 mM, an organic solvent, such as n-propanol, at a concentration between about 0-10% v/v, a chaotropic agent, such as urea, at a concentration of 0-8M, sodium citrate at a concentration of 0-8M, and/or a reducing agent, such as dithiothreitol (DTT), dithiobutylamine (DTBA), 2-mercaptoethanol (2-ME), or glutathione, at a concentration between about 0-50 mM, at a pH between about 4-12. In an exemplary embodiment, the lysis buffer can comprise 8M guanidine hydrochloride (Gu-HCl), 250 mM Tris-HCl, 2% n-propanol, and 50 mM dithiothreitol (DTT), at a pH of 8.6. In another exemplary embodiment, the lysis buffer can comprise 0.4M urea, 200 mM Tris-HCl, 25 mM sodium citrate, and 50 mM DTT, at pH of 7.4.

Without being bound to any theory, the forgoing formulation or composition can be optimal for RNA, DNA, and/or protein stability during heat lysis. In other embodiments, however, the lysis buffer formulation or composition can be sub-optimal for RNA, DNA, and/or protein stability during heat lysis. In particular, the optimal reagents, concentrations, etc. for lysis of DNA can be different than that for lysis of RNA, which can (each) be different than that for lysis of proteins. Accordingly, in certain embodiments, a user may (be required to) choose for which (proteogenomic) macromolecule to optimize the solution. In a preferred embodiment, the lysis buffer formulation or composition can be optimal for (enhancing stability of) RNA molecules in the sample.

In an embodiment, the deparaffinized FFPE (whole sections or LCM) tissue sample (from the 7 μm slice) can be mixed with approximately 0.5-1.0 ml (0.5 ml, 0.75 ml, 1.0 ml) of a lysis buffer solution. Other amounts are also contemplated herein and may depend on the thickness of the FFPE section.

In some embodiments, the lysis reaction can occur, takes place, and/or be performed at a particular temperature or between and/or within a particular temperature range. For instance, the lysis reaction temperature can be between about 25-95° C., preferably between about 55-85° C., more preferably between about 55-65° C., most preferably about 65° C. In some embodiments, the lysis reaction temperature can be less than about 80° C., 78° C., 75° C., 72° C., 70° C., 69° C., 68° C., 67° C., or 66° C. and/or greater than about 30° C., 32° C., 37° C., 42° C., 45° C., 50° C., 55° C., 60° C., 61° C., 62° C., 63° C., or 64° C.

In some embodiments, the lysis reaction can occur, takes place, and/or be performed for or over a particular time or time range. For instance, the lysis reaction time can be between about 0-2 hours, preferably between about 2 minutes to about 1 hour, more preferably between about 5 minutes to about 30 minutes, still more preferably between about 10 minutes to about 20 minutes, most preferably about 15 minutes. In at least one embodiment, the lysis reaction can be or comprise a single lysis step or period of time at a single lysis temperature or range.

In an embodiment, the lysis buffer can be or comprise (reagents found in) MagMAX™ kit lysis buffer commercially available from Thermo Fisher Scientific™.

In some embodiments, the lysis reaction can comprise a first lysis step at a first temperature and a second, subsequent lysis step at a second temperature. The first lysis step temperature can be between about 25-95° C., preferably between about 45-65° C., more preferably between about 50-60° C., most preferably about 55° C. In some embodiments, the first lysis step temperature can be less than about 80° C., 78° C., 75° C., 72° C., 70° C., 68° C., 65° C., 60° C., 58° C., or 56° C. and/or greater than about 30° C., 32° C., 37° C., 42° C., 45° C., 50° C., 52° C., or 54° C. The second lysis step temperature can be between about 25-95° C., preferably between about 65-90° C., more preferably between about 80-88° C., most preferably about 85° C. In some embodiments, the second lysis step temperature can be less than about 95° C., 92° C., 90° C., 88° C., or 86° C. and/or greater than about 30° C., 32° C., 37° C., 42° C., 45° C., 50° C., 55° C., 60° C., 65° C., 70° C., 75° C., 78° C., 80° C., 82° C., or 84° C.

In some embodiments, each step of the lysis reaction can occur, takes place, and/or be performed for or over a particular time or time range. For instance, the first lysis step time can be between about 0-2 hours, preferably between about 15 minutes to about 1.5 hours, more preferably between about 30 minutes to about 1.25 hours, still more preferably between about 45 minutes to about 1 hour, most preferably about 1 hour. The second lysis step time can be between about 0-2 hours, preferably between about 15 minutes to about 1.5 hours, more preferably between about 30 minutes to about 1.25 hours, still more preferably between about 45 minutes to about 1 hour, most preferably about 1 hour.

In an exemplary embodiment, the deparaffinized FFPE tissue can be mixed with approximately 0.5-1.0 ml of lysis buffer comprising 8M guanidine hydrochloride (Gu-HCl), 250 mM Tris-HCl, 2% n-propanol, and 50 mM dithiothreitol (DTT), at a pH of 8.6 and heated to 65° C. for exposure to the FFPE tissue section for a duration of approximately 15 minutes. In another exemplary embodiment, the lysis buffer can comprise 0.4M urea, 200 mM Tris-HCl, 25 mM sodium citrate, and 50 mM DTT, at pH of 7.4, heated to approximately 55° C. for exposure to an FFPE tissue section for approximately 1 hour and then heated to 85° C. for exposure to the tissue section for another hour. In yet another embodiment, the deparaffinized FFPE (whole sections or LCM) tissue sample (from the 7 μm slice) can be mixed with approximately 0.5-1.0 ml (0.5 ml, 0.75 ml, 1.0 ml) of MagMAX™ kit lysis buffer and heated at 55° C. for 1 hour and then at 85° C. for 1 hour. Other amounts are also contemplated herein and may depend on the thickness of the FFPE section.

Some embodiments can include a step 24 of alkylating proteins in the lysate. In at least one embodiment, alkylating proteins in the lysate can comprise adding an alkylating agent, such as iodoacetamide (IAM) or methyl methanethiosulfonate (MMTS), to the lysate. The alkylating agent can be added to the lysate at or to a concentration of between about 0-5 mM, preferably between about 1-5 mM, more preferably between about 2-4 mM, most preferably about 3.75 mM, depending on the agent used. For instance, an embodiment can include adding between about 1-10 μL, preferably between about 2-5 μL, more preferably about 3.75 μL of 1M IAM or MMTS (e.g., in 1M sodium bicarbonate, at a pH between about 8-12, preferably at a pH of 9) to the lysate. In at least one embodiment, the alkylation reaction can occur in the dark and/or at room temperature (or other suitable temperature) for a period of time between about 0-2 hours, preferably between about 5 minutes and about 1 hour, more preferably between about 10 minutes and about 45 minutes, still more preferably between about 15 minutes and about 30 minutes.

Some embodiments can include a step 26 of reducing alkylated proteins in the lysate. In at least one embodiment, reducing proteins in the lysate can comprise adding a reducing agent, such as dithiothreitol (DTT), tris(2-carboxyethyl)phosphine, dithiobutylamine (DTBA), 2-mercaptoethanol (2-ME), or glutathione, to the lysate. The reducing agent can be added to the lysate at or to a concentration of between about 0-50 mM, preferably between about 0.5-5 mM, more preferably between about 1-2 mM, most preferably about 1 mM, depending on the agent used. For instance, an embodiment can include adding between about 0-1000 μL, preferably between about 0.5-5 μL, more preferably about 1 μL of 1M DTT (or 0.5 μL of 2M DTT), to the lysate. In at least one embodiment, the reduction reaction can occur in the dark and/or at room temperature (or other suitable temperature) for a period of time between about 0-2 hours, preferably between about 5 minutes and about 1 hour, more preferably between about 10 minutes and about 45 minutes, still more preferably between about 15 minutes and about 30 minutes.

Some embodiments can include a step 28 of diluting alkylated and/or reduced proteins in the lysate. For instance, the lysate can be diluted with a dilution buffer or solution. The dilution buffer or solution can comprise, for example, 0-1000 mM Tris-HCl and 0-1000 mM CaCl₂ at a pH between about 4-10.0. A preferred embodiment can comprise diluting the lysate in (960 μL of) 50 mM Tris-HCl, 5 mM CaCl₂, with a suitable amount (e.g., 40 μL) of an RNase inactivation reagent, such as RNAsecure (commercially available from Thermo Fisher Scientific), at approximately pH 8.0.

Some embodiments can include a step 30 of enzymatically digesting alkylated and/or reduced proteins in the lysate. Without being bound to any theory, enzymatic digestion can be performed under conditions effective to release protein-bound RNA, DNA inside the nucleus, and cross-linked proteins, at quantities sufficient for downstream proteogenomic analysis. In at least one embodiment, enzymatically digesting proteins in the lysate can comprise incubating the lysate in the presence of a protease, such as trypsin, proteinase k, pepsin, etc. The protease can be added to the lysate at or to a concentration of between about 0-50 mM or final protease-to-protein ratio of 1:1 to 1:1000 (w/w), preferably 1:20 to 1:100 (w/w), more preferably 1:20, depending on the protease used and/or total protein concentration of the tissue section. In at least one embodiment, the digestion reaction can occur at between about 25° C. to 62° C., preferably between about 32° C. to 42° C., more preferably at about 37° C. and/or for a period of time between about 1-96 hours, preferably between about 4-24 hours, more preferably about 16 hours. The digestion reaction can be stopped by storing the samples at between about −20 to −80° C., preferably about −20° C. for 0.25-96 hours.

An embodiment can include reconstituting a 20 μg lyophilized stock of a MS-grade protease, such as trypsin, with between about 5-50 μL, preferably 20 μL of 0.01-1 M, preferably 50 mM acetic acid, adipic acid, malic acid, lactic acid, oxalic acid, malonic acid, succinic acid, glutaric acid, or picric acid, preferably acetic acid, to a concentration of between about 0.001-10 mg/mL, preferably about 1 mg/mL. The prepared protease enzyme can be used fresh or aliquoted into single use volumes and stored at −20 to −80° C., preferably about −80° C. Accordingly, proteins present in the lysate can be digested using a 1:20 ratio of MS grade trypsin (in 50 mM acetic acid) to total protein and incubated for approximately 16 hours at 37° C. with shaking. In one embodiment the trypsin can be immobilized trypsin for greater specificity and efficiency of protein digestion.

In at least one embodiment, the sample can be processed without exposing the proteins to any significant amount of sodium dodecyl sulfate (SDS), which can disrupt, interfere with, or perturb proteomic analysis, such as MS (e.g., by coating the protein and/or preventing ionization thereof). Processing samples without SDS can, however, pose a significant challenge to releasing and/or isolating DNA from inside the nucleus during lysis and/or digestion.

In at least one embodiment, the digestion step 30 can be performed with or using proteinase K, for example, in MagMAX™ or other buffer which may contain SDS. Without being bound to any theory, the use of proteinase K and/or SDS may release a larger quantity of DNA from the nucleus (as compared to tryptic digest and/or SDS-free processing), while released quantities of RNA and/or protein may be at least as high as with proteinase K digestion as with tryptic digestion. However, proteinase K digestion and/or SDS buffers may not be ideal for downstream proteomic analysis. Tryptic digest and/or guanidine HCl buffers can be more amendable to proteomic analysis. However, tryptic digest and/or guanidine HCl buffers may be less effective to release and/or isolate DNA during lysis and/or digestion. In addition, the extended time period that may be required to effectively release and/or isolate DNA using tryptic digest and/or guanidine HCl buffers may be detrimental to the stability of RNA in the reaction sample.

Embodiments of the present disclosure can reach a compromise between the need for robust DNA extraction, gentle RNA treatment, and protein analysis requirements. Such compromise-embodiments may not represent the most ideal reagents and/or reaction conditions for isolation of any of DNA, RNA, and/or proteins. However, certain compromise-embodiments can produce sufficient amounts of DNA, RNA and protein in suitable condition for downstream proteogenomic analysis, such as PCR, qRT-PCR, CGH, NGS, and/or MS (e.g., LC-MS).

Some embodiments can include a step 32 of separating nucleic acids (DNA and/or RNA) from digested proteins in the lysate and/or reaction sample. For instance, RNA, DNA and protein can be separated in the lysate or reaction sample using magnetic particle separation technology as is known in the art, preferably using an automated liquid handling system, such as the Kingfisher™ magnetic particle instrument and related kits (e.g., Kingfisher Pure RNA™ isolation kit), which are commercially available from Thermo Fisher Scientific.

By way of example, one or more aliquots of approximately 450 μL each can be removed from the reaction mixture (for each of RNA extraction and DNA extraction). RNase A or DNase I can be added to the aliquot, as applicable, for digestion of RNA (in the case of DNA isolation) or DNA (in the case of RNA isolation), respectively, as is known in the art. RNA or DNA can then be removed from the sample. By way of illustration, magnetic beads can be added to the reaction sample. The beads can bind free nucleic acids (NA), or vice versa, from the lysate. A magnetic rod or other element can remove the NA-bound magnetic beads, which can be washed (e.g., with alcohol and/or proprietary wash buffer). NA can then be eluted from the beads (e.g., with (nuclease-free) water and/or proprietary elution buffer) and prepared for downstream assays (e.g., PCR, RTqPCR, microarray, CGH, and/or NGS). In some embodiments, 25-100 μl, preferably 50 μL of NA can be eluted for each aliquot.

Some embodiments can include a step 34 of analyzing separated nucleic acids (DNA and/or RNA). RNA and/or DNA can be quantified, for example, with a Qubit® fluorometer (commercially available from Thermo Fisher Scientific) to quantitate the amount of NA in the sample, a bioanalyzer instrument (for example, the Agilent™ 2100 bioanalyzer commercially available from Agilent Technologies) to detect fragment NA, and/or a NanoDrop™ 2000c spectrophotometer (commercially available from Thermo Fisher Scientific) to measure the relative purity of the sample.

After quantification, RNA and DNA can be analyzed through analytical procedures including amplification (via PCR, qPCR, RTqPCR, etc.) and next-generation sequencing (NGS), as are known in the art. Genomic analysis (via NGS) can be performed using the Ion Torrent™ Personal Gene Machine™ (PGM) instrument, which is commercially available from Thermo Fisher Scientific, using kits designed for use with the PGM instrument (e.g., AmpliSeq™ Cancer Hotspot panel products, which target 50 genes available from Thermo Fisher Scientific).

Proteins can also be recovered from the lysate or reaction mixture. For instance, at least a portion of the remaining lysate or reaction sample (after taking aliquots for NA isolation, purification, and/or analysis) can be processed for protein recovery. Proteins can also (or alternatively) be recovered from one or more of the DNA and/or RNA aliquots (e.g., after magnetic removal of NA). In at least one embodiment, the remaining lysate or reaction sample can be combined with the separate DNA and RNA aliquot residues and prepared for subsequent purification and protein analysis by liquid chromatography mass spectrometry (LC-MS). The combined RNA and DNA residues can provide between about 900-1800 μL of sample and the original, unused protease digested lysate can provide about 100 μL of sample, in certain embodiments.

Some embodiments can include a step 36 of analyzing proteins and/or peptides, as known in the art. In certain embodiments, the analysis can include LC-MS. By way of example, single or combined samples can be dried, for example using a vacuum concentrator (e.g., Speedvac™ vacuum concentrator, commercially available from Thermo Fisher Scientific). The dried sample can then be brought to a final volume of 1 mL using 0.1% formic acid in LC-MS grade water, as known in the art. Peptides can be further purified and concentrated by solid phase extraction using C4, C12, or C18 resin in cartridges or plates for example, a HyperSep™ Retain CX (30 mg) 96-well plate, commercially available from Thermo Fisher Scientific. Plates can be conditioned with 1 mL of 1% ammonium hydroxide, 75% isopropyl alcohol in LC-MS grade water and applying vacuum pressure. Wells can be equilibrated with 1 mL of 0.1% formic acid in LC-MS grade water and applying vacuum pressure. Plates can again be conditioned with 1 mL of 1% ammonium hydroxide, 75% isopropyl alcohol in LC-MS grade water and applying vacuum pressure.

In some embodiments, 1 mL of the prepared peptide sample can be loaded into a conditioned and equilibrated well. In a high throughput system, multiple prepared peptide samples can be loaded, respectively, into separate conditioned and equilibrated wells. Vacuum pressure can be applied to run the samples through the well(s). Well(s) can be washed with 1 mL of 0.1% formic acid in LC-MS grade water and washed (e.g., twice) with 1 mL of 10-100% isopropyl alcohol (IPA), preferably 10% IPA, in 0.1% formic acid.

Peptides can be eluted using 100 uL of 1% ammonium hydroxide, 75% isopropyl alcohol in LC-MS grade water (e.g., three times). Eluted peptide samples can be concentrated to dryness, re-suspended in 25 uL of 0.1% formic acid in water, and analyzed by HPLC/MS in discovery or targeted mass spectrometry modes. Proteomic (MS) analysis can be conducted using the Q-Exactive™ mass spectrometer (commercially available from Thermo Fisher Scientific).

The foregoing and other methods can enable users to isolate RNA, DNA, and protein from the same section, piece, and/or quadrant of formalin-fixed, paraffin-embedded (FFPE) tissue. When combined with laser-capture microdissection (LCM), some embodiments can enable users to correlate RNA, DNA, and protein status and/or characteristics from the same portion of a tissue. The risk of obtaining misleading or conflicting genomic and proteomic data can thereby be decreased (because (proteogenomic material from) the same section and/or same cells are involved in the analysis). Further, because a single thin section (approximately 7 micron) can be used for both nucleic acid and protein analytics, the remainder of the FFPE tissue block can be available for further analysis as may be needed for later studies.

In at least one embodiment, one or more of the foregoing or other apparatus, reagents, kits, etc. can be (fluid) coupled, combined, and/or connected to form a (single, stand-alone) system for extraction, preparation, isolation, and/or proteogenomic analysis of one or more biological molecules (e.g., nucleic acid, such as DNA and/or RNA, proteins and/or peptides, etc.). Such systems can provide efficient and cost effective means for conducting proteogenomic analysis for a variety of intended purposes. By way of example, systems, methods, and/or products of the present disclosure can be useful in differentiating cancer subtypes. Accordingly, certain embodiments of the present disclosure can include systems, methods, and/or products for differentiating cancer subtypes. Such embodiments can include, comprise, and/or incorporate one or more of the foregoing or other apparatus, reagents, kits, methods, steps, etc.

One or more embodiments can include a peptide panel. The panel can comprise a plurality of peptides for identifying the presence of one or more proteins in a sample, such as a FFPE tissue section, differentiating between cancer subtypes (associated with the identified proteins), and/or measuring level of expression of drug targets. In at least one embodiment, proteins indicative of certain cancers or cancer subtypes can be identified, (quantitatively) measured, or determined to be present in a sample by detecting one or more peptides of the proteins.

By way of example, the specific form of the proteins MET, EGFR, HER2 and KRAS in a cancerous (e.g., lung or breast) tissue that has been biopsied and prepared as a FFPE tissue sample can be determined through implementation of one or more embodiments of the present disclosure. Such a determination can be useful for differentiating between (lung or breast) cancer subtypes (e.g., squamous, adenocarcinoma, etc.) and discovering the level of expression of these proteins (i.e., potential drug targets).

The panel can include a suitable number of peptides for identifying a suitable number (e.g., between about 3-5, 7-9, 10-12, etc.) of protein variants indicative of a particular cancer type. Each peptide can have one or more, two or more, a plurality, at least 3, at least 4, or at least 5 transition ions. An illustrative panel of peptides is illustrated in the listing below. The listing includes a variety of peptides, any suitable number of which may be useful for identifying protein variants indicative of a particular cancer type, such as breast or lung cancer, as indicated below:

| Protein Name | Peptide Sequence |
|---|---|
| BREAST | |
| 4E-BP1_1 | HYDRKFL(Met[O])EC(CAM)RNSPVTKTPP(R) |
| 4E-BP1_2 | KFLMEC(R) |
| 4E-BP1_3 | NSPVTKTPP(R) |
| 4E-BP1_4 | FLMEC(R) |
| AKT_1 | DLKLENLMLDKDGHI(K) |
| AKT_2 | EGWLHKRGEYIKTWRP(R) |
| AKT_3 | ATGRYYAM(K) |
| AKT_4 | LPFYNQDHE(K) |
| AKT_5 | KLSPPFKPQVTSETDT(R) |
| AKT_6 | KEVIVAKDEVAHTLTEN(R) |
| AKT_7 | HPFLTALKYSFQTHD(R) |
| AKT_8 | ERVFSEDRA(R) |
| AR_1 | MYSQC(CAM)V(R) |
| AR_2 | QLVHVV(K) |
| AR_3 | RFYQLTKLLDSVQPIA(R) |
| AR_4 | GAFQNLFQSVREVIQNPGP(R) |
| AR_5 | FFDEL(R) |
| AR_6 | SFTNVNSRMLYFAPDLVFNEY(R) |
| AR_7 | SHMVSVDFPEMMAEIISVQVP(K) |
| BRAF_1 | SNPKSPQKPIVRVFLPNKQ(R) |
| BRAF_10 | RLMAEC(CAM)LK(K) |
| BRAF_2 | LLFQGF(R) |
| BRAF_3 | DLKSNNIFLHEDLTV(K) |
| BRAF_4 | DQIIFMVGRGYLSPDLSKV(R) |
| BRAF_5 | TFFTLAFC(CAM)DFC(CAM)(R) |
| BRAF_6 | LDALQQ(R) |
| BRAF_7 | C(CAM)GVTVRDSLK(K) |
| BRAF_8 | GLIPEC(CAM)C(CAM)AVY(R) |
| BRAF_9 | QTAQGMDYLHA(K) |
| Caspase3_1 | SGTDVDAANL(R) |
| Caspase3_2 | LFIIQAC(R) |
| Caspase6_1 | IFIIQAC(CAM)(R) |
| Caspase6_2 | FSDLGFEV(K) |
| Caspase6_3 | RGIALIFNHE(R) |
| Caspase6_4 | GNQHDVPVIPLDVVDNQTE(K) |
| Caspase6_5 | EMFDPAE(K) |
| Caspase6_6 | GHPAGGEENMTETDAFY(K) |
| Caspase8_1 | V(Met[O])LYQISEEVSRSEL(R) |
| Caspase8_2 | RVC(CAM)AQIN(K) |
| Caspase8_3 | GDDILTILTEVNYEVSNKDDK(K) |
| Caspase8_4 | QMPQPTFTLR(K) |
| Caspase9_1 | TRTGSNIDC(CAM)EKL(R) |
| Caspase9_2 | IVNIFNGTSC(CAM)PSLGGKP(K) |
| Caspase9_3 | QMPGC(CAM)FNFL(R) |
| Caspase9_4 | LSKPTLENLTPVVLRPEI(R) |
| Caspase9_5 | QLIIDLET(R) |
| cMyc_1 | LASYQAAR(K) |
| cMyc_2 | VKLDSV(R) |
| cMyc_3 | SSDTEENVKRRTHNVLE(R) |
| cMyc_4 | DQIPELENNEKAP(K) |
| cMyc_5 | HKLEQL(R) |
| cMyc_6 | KATAYILSVQAEEQKLISEEDLLR(K) |
| CTLA4_1 | A(Met[O])HVAQPAVVLASS(R) |
| CTLA4_2 | A(Met[O])DTGLYIC(CAM)(K) |
| ER_1 | EAGPPAFYRPNSDNR(R) |
| ER_2 | LASTNDKGSMAMESAKET(R) |
| ER_3 | QRDDGEGRGEVGSAGDM(R) |
| ER_4 | LLFAPNLLLD(R) |
| ER_5 | KC(CAM)YEVGMM(K) |
| ER_6 | RSIQGNRHNDY[Met(O)]CPATNQCTID(K) |
| ER_7 | SIQGHNDY[Met(O)]C(CAM)PATNQC(CAM) |
| | TIDKNR(R) |
| ERK_1 | IADPEHDHTGFLTEYVAT(R) |
| ERK_2 | FRHENVIGI(R) |
| ERK_3 | EIQILL(R) |
| ERK_4 | NYLQSLPS(K) |
| ERK_5 | ALDLLD(R) |
| ERK_6 | TKVAWA(K) |
| ERK_7 | IC(CAM)DFGLA(R) |
| ERK_8 | LFPKSDS(K) |
| FGFR1_1 | NGKEFKPDH(R) |
| FGFR1_2 | TSNRGHKVEVSWEQ(R) |
| FGFR1_3 | FKC(CAM)PSSGTPNPTL(R) |
| FGFR2_1 | GATPRDSGLYACTAS(R) |
| FGFR4_1 | HQHWSLVMESVVPSD(R) |
| MAPK_1 | VADPDHDHTGFLTEYVAT(R) |
| MAPK_2 | DLKPSNLLLNTTC(CAM)DL(K) |
| MAPK_3 | LFPNADS(K) |
| MAPK_4 | GQVFDVGP(R) |
| MAPK_5 | APEI(Met[0])LNS(K) |
| MAPK_6 | LKELIFEETA(R) |
| MEK1_1 | ISELGAGNGGVVF(K) |
| MEK1_2 | IPEQILG(K) |
| MEK1_3 | DVKPSNILVNS(R) |
| MEK1_4 | SYMSPE(R) |
| mTOR_1 | TLDQSPEL(R) |
| mTOR_10 | DFSHDDTLDVPTQVELLI(K) |
| mTOR_2 | WTLVNDETQAKMA(R) |
| mTOR_3 | LAMAGDTFTAEYVEFEV(K) |
| mTOR_4 | STAMDTLSSLVFQLG(K) |
| mTOR_5 | LMDTNTKGNK(R) |
| mTOR_6 | ELQHYVTMEL(R) |
| mTOR_7 | HC(CAM)ADHFLNSEHKEI(R) |
| mTOR_8 | IVEDWQ(K) |
| mTOR_9 | GNNLQDTL(R) |
| NFkB-p100_1 | QTTSPSGSLL(R) |
| NFkB-p65_1 | APNTAELKIC(CAM)(R) |
| NFkB-p65_2 | NSGSC(CAM)LGGDEIFLLC(CAM)D(K) |
| NFkB-p65_3 | KRTYETF(K) |
| NFkB-p65_4 | TPPYADPSLQAPV(R) |
| NFkB-p65_5 | LPPVLSHPIFDN(R) |
| NFkB-p65_6 | KSPFSGPTDPRPPPR(R) |
| NFkB-relB_1 | KEIEAAIE(R) |
| NFkB-relB_2 | IQLGIDPYNAGSL(K) |
| NFkB-relB_3 | EDISVVFSRASWEG(R) |
| PCNA_1 | LVQGSIL(K) |
| PCNA_2 | C(CAM)AGNEDIITL(R) |
| PCNA_3 | VSDYEM(K) |
| PCNA_4 | DLSHIGDAVVISCA(K) |
| PCNA_5 | FSASGELGNGNI(K) |
| PCNA_6 | SEGFDTYRC(CAM)D(R) |
| PCNA_7 | [Met(O)]PSGEFA(R) |
| PDL1_1 | LFNVTSTLRINTTTNEIFYC(CAM)TF(R) |
| PDL1_2 | LQDAGVY(R) |
| PDL1_3 | LFNVTSTL(R) |
| PDL1_4 | VNAPYN(K) |
| PDL1_5 | CMISYGGADY(K) |
| PI3K_1 | LNTEETVKVHV(R) |
| PI3K_2 | ALETSVAADFYH(R) |
| PI3K_3 | DHESVFTVSLWDC(CAM)DR(K) |
| PI3K_4 | FEPYHDSALA(R) |
| PI3K_5 | SFLGINKE(R) |
| PI3K_6 | YQVVQTLDC(CAM)L(R) |
| PI3K_7 | MAEVASRDP(K) |
| PI3K_8 | KTSPHFQKFQDIC(CAM)V(K) |
| PR_1 | TQDQQSLSDVEGAYS(R) |
| PR_2 | KC(CAM)C(CAM)QAGMVLGGR(K) |
| PR_3 | FYQLTKLLDNLHDLV(K) |
| PR_4 | ALSVEFPE(Met[O])(Met[O])SEVIAAQLP |
| | (K) |
| PR_5 | SSYIRELI(K) |
| PR_6 | RA[Met(O)]EGQHNYLC(CAM)AGRNDC(CAM) |
| | IVDKIR(R) |
| PR_7 | ALDAVALPQPVGVPNESQALSQ(R) |
| PR_8 | SYKHVSGQMLYFAPDLILNEQ(R) |
| PTEN_1 | IYNLC(CAM)AERHYDTAKFNC(CAM)(R) |
| PTEN_2 | AQEALDFYGEV(R) |
| PTEN_3 | DKKGVTIPSQR(R) |
| PTEN_4 | VKIYSSNSGPT(R) |
| PTEN_5 | YFSPNF(K) |
| PTEN_6 | NNIDDVV(R) |
| PTEN_7 | ADNDKEYLVLTLTKNDLD(K) |
| rhoA_1 | ISAFGYLEC(CAM)SA(K) |
| rhoA_6 | FKRFPCLSLLSSWGY(R) |
| rhoAC_1 | EVFE(Met[O])AT(R) |
| rhoAC_2 | HFC(CAM)PNVPIILVGNK(K) |
| rhoAC_3 | KKLVIVGDGAC(CAM)G(K) |
| rhoC_1 | IGAFGYMECSA(K) |
| rhoC_2 | QVELALWDTAGQEDYD(R) |
| rhoC_3 | DGVREVFEMATRAALQA(R) |
| S_6K_1 | LGAGPGDAGEVQAHPFF(R) |
| S_6K_2 | FSLSGGYWNSVSDTA(K) |
| S_6K_3 | LTAALVL(R) |
| S_6K_4 | HPWIVHWDQLPQYQLN(R) |
| S_6K_5 | DSPGIPPSANAHQLF(R) |
| LUNG | |
| CK5_1 | TSFTSVS(R) |
| CK5_2 | YEELQQTAG(R) |
| CK5_3 | AQYEEIAN(R) |
| CK5_4 | EYQELMNT(K) |
| CK5_5 | FVSTTSSS(R) |
| CK6_1 | EYQELMNV(K) |
| CK6_2 | TAAENEFVTL(K) |
| CK6_3 | EELQVTAG(R) |
| CK6_4 | SGFSSISVS(R) |
| CK6_5 | ATGGGLSSVGGGSSTI(K) |
| CK7_1 | LDADPSLQ(R) |
| CK7_2 | GQLEALQVDGG(R) |
| CK7_3 | DVDAAYMS(K) |
| CK7_4 | NEISEMN(R) |
| CK7_5 | LLEGEES(R) |
| CK20_1 | QWYETNAP(R) |
| CK20_2 | LEQEIATY(R) |
| CK20_3 | TTEYQLSTLEE(R) |
| CK20_4 | TVVQEVVDG(K) |
| CK20_5 | VLQIDNAKLAAEDF(R) |
| MET_1 | DLGSELV(R) |
| MET_2 | SVSPTTEMVSNESVDY(R) |
| MET_2_pY1003 | SVSPTTEMVSNESVD[Y](R) |
| MET_3_L1213L | N(CAM)MLDE(K) |
| MET_3_L1213V | N(CAM)MVDE(K) |
| MET_4_Y1248Y | DMYDKEYYSVHN(K) |
| MET_4_Y1248H | DMHDKEYYSVHN(K) |
| MET_4_ | DMYDKE[Y]YSVHN(K) |
| Y1248Y_pY1234 | |
| MET_4_ | DMYDKEY[Y]SVHN(K) |
| Y1248Y_pY1235 | |
| MET_4_Y1248Y_ | DMYDKE[Y][Y]SVHN(K) |
| pY1234_pY1235 | |
| MET_5_M1268M | WMALESLQTQ(K) |
| MET_5_M1268T | WTALESLQTQ(K) |
| EGFR_1 | YSFGAT(CAM)V(K) |
| EGFR_2 | V(CAM)NGIGIGEF(K) |
| EGFR_3 | N(CAM)TSISGDLHILPVAF(R) |
| HER2_1 | DPPFC(CAM)VA(R) |
| HER2_2 | GMSYLEDV(R) |
| HER2_3 | ELVSEFS(R) |
| HER2_4 | SGGGDLTLGLEPSEEEAP(R) |
| HER2_4_pS_ | [S]GGGDLTLGLEPSEEEAP(R) |
| 1051 | |
| HER2_4_pS_ | SGGGDLTLGLEP[S]EEEAP(R) |
| 1054 | |
| HER2_4_pS_ | [S]GGGDLTLGLEP[S]EEEAP(R) |
| 1051_pS_1054 | |
| HER2_5 | GLQSLPTHDPSPLQ(R) |
| HER2_5_pS_ | GLQ[S]LPTHDPSPLQ(R) |
| 1100 | |
| HER2_5_pS_ | GLQSLPTHDP[S]PLQ(R) |
| 1007 | |
| HER2_5_pS_ | GLQ[S]LPTHDP[S]PLQ(R) |
| 1100_pS_1007 | |
| KRAS_1 | LVVVGAGGVG(K) |
| KRAS_2A | VKDSEDVPMVLVGN(K) |
| KRAS_2B | DSEDVPMVLVGN(K) |
| KRAS_3 | SYGIPFIETSA(K) |
| KRAS_4 | QGVDDAFYTLV(R) |
| NAPSINA_1A | FAIQYGTGRVDGILSED(K) |
| NAPSINA_1B | VDGILSED(K) |
| NAPSINA_1C | FAIQYGTG(R) |
| NAPSINA_2 | VGPGLTL(CAM)A(K) |
| P40/63_1 | SATWTYSTEL(K) |
| P40/63_2 | EFNEGQIAPPSHLI(R) |
| P40/63_3 | ICA(CAM)PG(R) |
| P40/63_4 | ETYEMLL(K) |
| P40/63_S | TPSSASTVSVGSSET(R) |

The above listing incorporates established single-letter convention for amino acid residues and punctuation convention for modification thereof. Thus, the above listing corresponds as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), valine (V). Moreover, deuterated residues (lysine and/or arginine) are indicated by parenthesis; (X), phosphorylated residues (serine and/or tyrosine) are indicated by brackets; [X], oxidized Methionine residues—i.e., Methionine sulfoxide—are indicated by the designation [Met(O)] or (Met[O]), and carbamidomethylation (CAM) modifications are indicated by the designation (CAM) following the modified amino acid residue.

A method of differentiating between cancer subtypes can include or incorporate one or more of the foregoing systems, methods, and/or products, or parts, steps, or components thereof. The method can include detecting one or more of the peptides (fragments) listed above in a biological tissue sample. Detection can include performing MS analysis (as described herein). The method can include identifying one or more of the protein variants corresponding with the peptides and/or searching a database to determine a cancer or cancer subtype known to express the identified protein(s) or peptides. The method can be performed automatically by certain embodiments of the present disclosure.

The relative quantity of detected proteins compared to housekeeping proteins may be determined. When digested peptide samples are run in discovery mode in LC-MS, peak area for each of the individual peptides is determined. Detected proteins are relatively quantified by comparing the average of each target protein peak area to the average of a housekeeping protein's peak areas. To determine the appropriate normalizing housekeeping protein, the total ion count for each sample is compared to the average of the highest ranked housekeeping protein peptides >n=10 sorted by delta score and then Xcorr value. The selected housekeeping protein is selected by the smallest standard deviation in comparison to the total ion count. Suitable housekeeping proteins may include: GAPDH, βACTIN, RPSL11, TUBA1A, TUBA1B and others. The same process is used for selecting the target protein peptides for relative quantitation. The averaged peak area of each protein divided by the averaged peak area of the house keeping proteins provides the relative expression value.

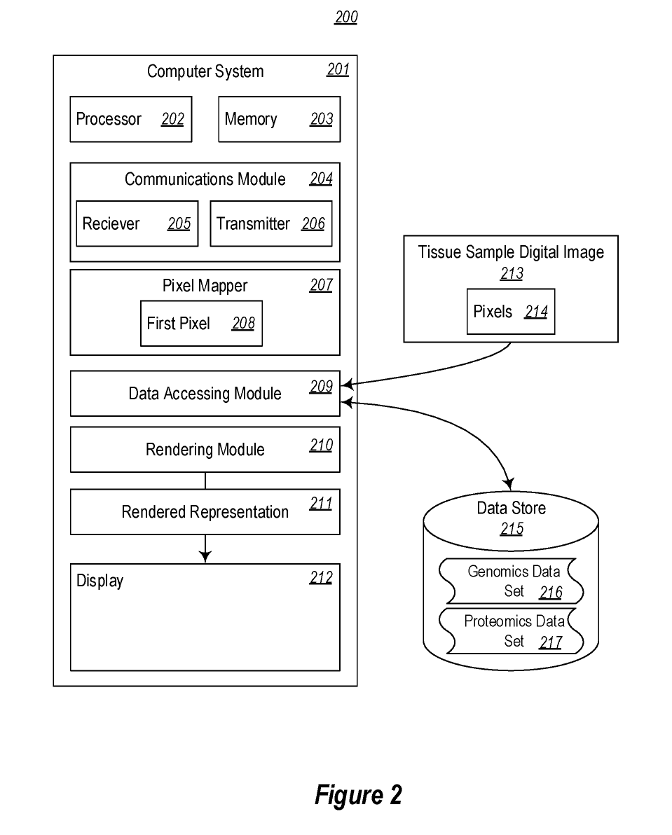

Turning now to FIG. 2, a computing environment 200 is shown in which various embodiments may be implemented. The computing environment 200 includes at least one computer system 201. The computer system 201 can be any type of computer system that has a processor 202 and memory 203. Indeed, it will be recognized that computer systems are now increasingly taking a wide variety of forms. Computer systems may, for example, be mobile phones, electronic appliances, laptop computers, tablet computers, wearable devices, desktop computers, mainframes, and the like. As used herein, the term “computer system” includes any device, system, or combination thereof that includes at least one processor, and a physical and tangible computer-readable memory capable of having thereon computer-executable instructions that are executable by the processor.

A computing system may be distributed over a network environment and may include multiple constituent computing systems (e.g., a cloud computing environment). In a cloud computing environment, program modules may be located in both local and remote memory storage devices. As described herein, a computer system may also contain communication channels that allow the computing system to communicate with other message processors over a wired or wireless network. Such communication channels may include hardware-based receivers (e.g., 205), transmitters (e.g., 206), or transceivers, which are configured to receive data, transmit data or perform both.

Embodiments described herein also include physical computer-readable media for carrying or storing computer-executable instructions and/or data structures. Such computer-readable media can be any available physical media that can be accessed by a general-purpose or special-purpose computing system. Still further, system architectures described herein can include a plurality of independent components that each contribute to the functionality of the system as a whole. This modularity allows for increased flexibility when approaching issues of platform scalability and, to this end, provides a variety of advantages. System complexity and growth can be managed more easily through the use of smaller-scale parts with limited functional scope. Platform fault tolerance is enhanced through the use of these loosely coupled modules. Individual components can be grown incrementally as business needs dictate. Modular development also translates to decreased time to market for new functionality. New functionality can be added or subtracted without impacting the core system.

The computer system 201 may include many different modules or components configured to perform certain functions. For instance, the communications module 204 may be configured to receive and/or transmit data. The pixel mapper 207 may be configured to access digital images and identify certain pixels within those images. For example, pixel mapper 207 may access tissue sample digital image 213 and identify pixels within that image. The tissue sample digital image 213 may be the result of or a representation of the laser capture microdissect FFPE 20 tissue sample from FIG. 1. The tissue sample digital image 213 includes many different pixels 214 that represent the image of the tissue. The pixel mapper 207 identifies pixels in the digital image (e.g., first pixel 208), and maps those pixels to a portion of the tissue sample. Accordingly, the pixel mapper 207 is configured to look at a digital image and map individual pixels of that image to a tissue sample. This mapping may then be used when generating a representation of genomics and proteomics data 216 and 217.

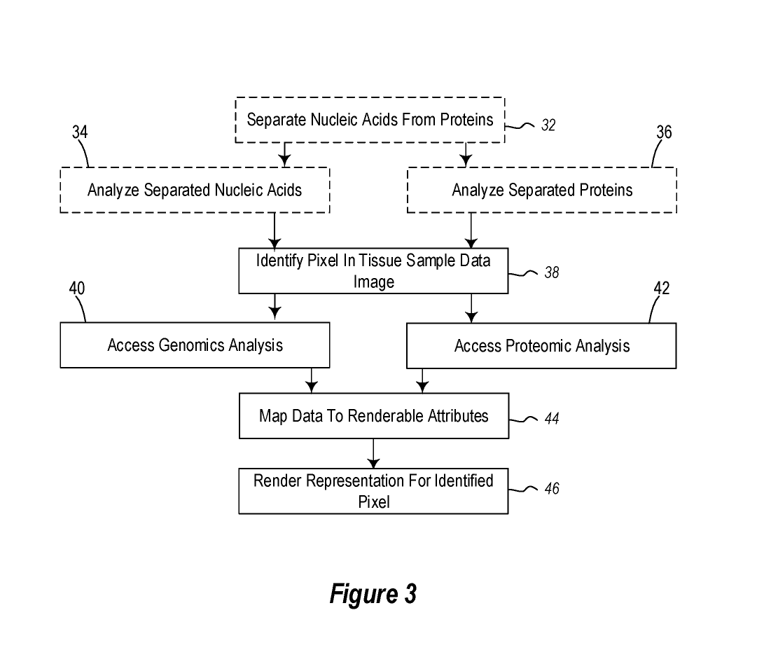

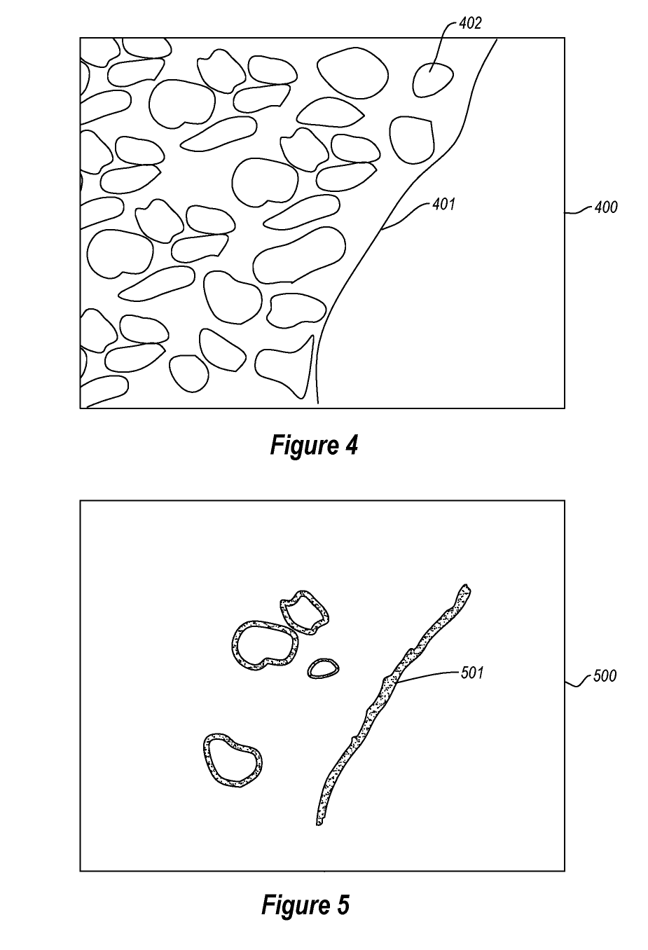

The data accessing module 209 of computer system 201 may access genomics data 216 and proteomics data 217 from a local or remote data store 215. The genomics data 216 may be the result of analysis step 34 of FIG. 1, while the proteomics data 217 may be the result of analysis step 36 of FIG. 1. These data sets may be used, in conjunction with the pixel mapping information, to create a rendered representation 211. Each data set may have attributes which are renderable in a representation. The rendering module 210 can identify these renderable attributes and generate a part of a representation based on those attributes. The part of the representation may be a single pixel or a group of pixels. The rendering module 210 may thus render a new representation based on the renderable attributes one pixel at a time, according to the pixel mapping. Once the rendered representation 211 (or at least a portion thereof) has been generated, it can be shown on display 212, or broadcast to another display. The rendered representation can thus show certain attributes related to the data sets 216 and 217. In one example, the representation may be a heat map, showing the relative presence or absence of nucleic acids or proteins in the tissue sample. These concepts will be explained further below with regard to the flow diagram of FIG. 3, along with additional FIGS. 4-7.

Some embodiments include a system, such as a system for generating a representation of cell content of a tissue sample on a pixel-by-pixel basis. Some embodiments can include a nucleic acid analyzing element and/or a protein analyzing element, as described in further detail above. The nucleic acid analyzing element can be configured to produce a first data set. The first data set can represent a genomic analysis of one or more nucleic acids. The genomic analysis can be performed at least in part by the nucleic acid analyzing element. The nucleic acid analyzing element can comprise one or more apparatus. The one or more apparatus can, for example, include or be selected from the group consisting of nucleic acid quantification devices (e.g., a fluorometer), nucleic acid fragment detection devices (e.g., a bioanalyzer instrument), nucleic acid purity measurement devices (e.g., a spectrophotometer), nucleic acid amplification devices (e.g., a thermal cycler), and nucleic acid sequencing devices (e.g., a (next-generation) sequencer).

The protein analyzing element can be configured to produce a second data set. The second data set can represent a proteomic analysis of one or more proteins. The proteomic analysis can be performed at least in part by the protein analyzing element. The protein analyzing element can comprise one or more apparatus. The one or more apparatus can include or be selected from the group consisting of protein purification devices (e.g., a (high performance and/or liquid) chromatography device, a (solid phase) extraction device, such as a cartridge or plate, etc.) and mass spectrometry devices (e.g., mass spectrometer or mass spectrometry system). Some embodiments can include a tandem or fluid coupled liquid chromatography mass spectrometry (LC-MS) system.

Some embodiments can include a tissue sample processing system comprising one or more sample receptacles and reagents for processing a tissue sample (e.g., a cell lysing reagent, a protein alkylating reagent, a protein reducing reagent, a protein digesting reagent, etc.). Some embodiments can include a sample concentrating element (e.g., vacuum concentrator). In some embodiments, the tissue sample processing system and/or sample concentrating element, or one or more respective components thereof, can be fluid coupled with the nucleic acid analyzing element and/or protein analyzing element, or one or more respective components thereof.

Some embodiments can include a pixel mapper. The pixel mapper can be configured to identify a first pixel in a digital image of a tissue sample. The identified first pixel can be mapped to or onto a portion of the tissue sample or digital image thereof.

Some embodiments can include a data accessing module. The data accessing module can be configured to access the first data set and/or the second data set.

Some embodiments can include a processor. The processor can be configured to perform one or more steps or actions. In some embodiments, for instance, the processor can be configured to map the accessed first data set and/or the second data set to a renderable attribute. The renderable attribute can (thereby) have a specified value based on the mapped data sets. In some embodiments, the processor can be configured to render at least a portion of a representation. The representation can correspond to the identified first pixel. The rendering can be done according to the specified value of the renderable attribute. The representation can include an indication of how one or more nucleic acids and/or one or more proteins are distributed within the tissue sample.

FIG. 3 illustrates a flowchart of a method for representing cell content of a tissue sample. FIG. 3 initially shows steps 32, 34 and 36 from FIG. 1. These steps may be performed at some point prior to the performance of FIG. 3. In step 38 of FIG. 3, the pixel mapper 207 of FIG. 2 identifies a first pixel 208 in a digital image of a tissue sample (e.g., image 213). This first pixel 208 is mapped to a portion of the tissue sample. The pixel mapping may indicate that the pixel corresponds to or is the representation of that part of the tissue sample. Each pixel of the image may thus, in combination with the other pixels, form an image of the tissue.

The data accessing module 209 then accesses a first data set 216 representing a genomics analysis of nucleic acids that are separated from tissue proteins in the tissue sample (step 40), and further accesses a second data set 217 representing a proteomic analysis of proteins that were separated from the nucleic acids of the tissue sample (step 42). This data may be stored locally in a local data store, or may be stored remotely in a remote data store. Regardless, the data sets are accessed from data store 215 and are used in step 44 to map the accessed first and second data sets (216, 217) to a renderable attribute. This renderable attribute may be any type of characteristic or feature related to the tissue sample. For instance, the tissue sample 401 of FIG. 4 may show individual cells 402. The tissue sample 401 may show nucleic acids (or indicators thereof), proteins (or indicators thereof), or other biological features. Each of these may be a renderable attribute, and each renderable attribute may have a specified value based on the mapped data sets.

For example, the genomics data set 216 may include data regarding the presence (or characteristics) of nucleic acids in the tissue sample. Proteomics data 217, on the other hand, may include data regarding the presence (or characteristics) of proteins in the tissue sample. In cases where the renderable attribute comprises the presence of nucleic acids and proteins in the tissue sample, the amount of nucleic acids or proteins at a given location (represented by a pixel) may be associated with a specified value. This value may, in turn, be associated with a color which can be rendered on top of the image as an overlay. Collectively, then, this overlay can show a heat map, indicating where an increased amount of nucleic acids and/or proteins are located on the tissue sample. An example of such a heat map overlay 500 is shown in FIG. 5. FIG. 5 shows a differential or “diff” image that identifies the differences 501 between the initial tissue sample image 400 and the newly rendered heat map overlay 500. When the heat map overlay 500 is positioned on top of the tissue sample image 400, the rendering can show where on the tissue sample the different nucleic acids or proteins are grouped.

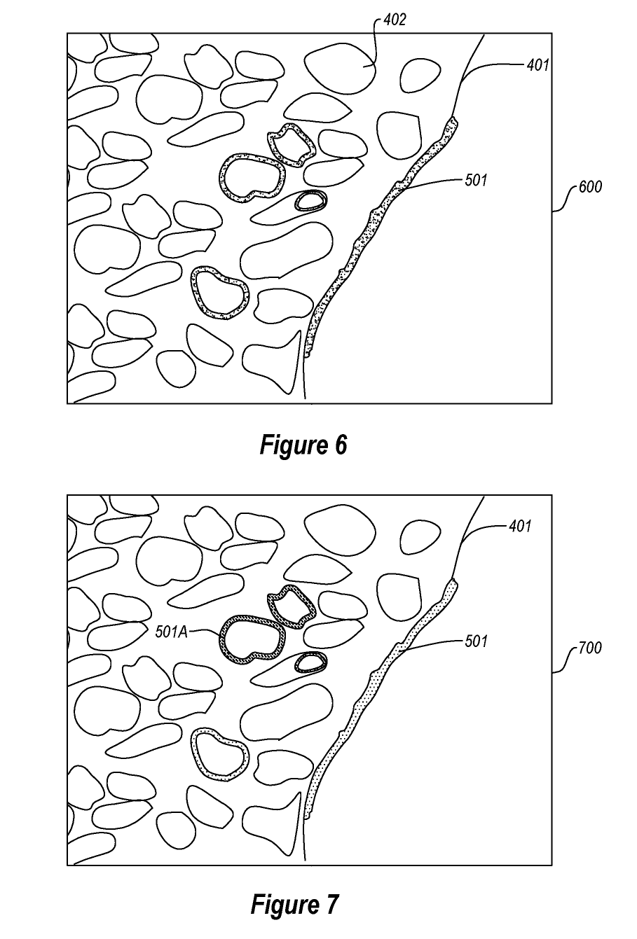

For instance, image 600 of FIG. 6 shows the tissue sample 401, along with its cells 402, as well as the heat map overlay 500 of FIG. 5 which shows where the nucleic acids and proteins are grouped (as illustrated by the areas filled with a dot pattern). As can be seen in image 700 of FIG. 7, the nucleic acid groupings and the protein groupings may be illustrated in different colors (or different dot patterns 501 and 501A). It will be understood to one skilled in the art that substantially any type of biological feature or characteristic may be represented in the overlay, and that the overlay may use any number of layers or colors or patterns to illustrate how that biological feature relates to the underling image.

Accordingly, in the method described in FIG. 3 above, for each pixel 214 in the tissue sample digital image 213, the rendering module 210 can render at least a portion (e.g., a pixel-sized portion) of a new representation 211 corresponding to the identified first pixel 208. The pixel-sized portion is rendered according to the specified value of the renderable attribute. Thus, if the renderable attribute is an indication of how many nucleic acids are present at that pixel, or how many proteins are present at that pixel, the renderable attribute will have or will be assigned a value based on the data sets (216, 217), and that value can be represented in the overlay. This (pixel-sized) portion of the representation corresponding to the first pixel can then be superimposed over the first pixel 208 of the tissue sample digital image. When this occurs over multiple pixels, a pattern can be seen, such as the pattern in FIGS. 5-7. The pattern in the overlay superimposed over the pixels of the tissue sample digital image 213 shows a heat map representing (or representative of) the relative presence of separated nucleic acids and proteins (provided from data sets 216 and 217).

In some cases, the data sets 216 and 217 are filtered lists of data. In such cases, the data is filtered according to user-defined parameters. The user may indicate, for example, which nucleic acids or proteins or other biological features are to be provided in the list. Non-selected features are left out of the data sets used in the rendering.

As noted in FIG. 1, performing a laser capture microdissect on a formalin-fixed paraffin-embedded (FFPE) tissue sample may be part of an overall process in which a tissue sample is imaged and then overlain with a new data representation. In cases where the tissue sample is a laser capture microdissect FFPE tissue section, the digital image shows a portion of FFPE tissue that is remaining after the laser capture microdissect has been performed. Data is gathered for each cell or group of cells, and may be mapped to pixels of a heat map. This may result in an image with four cut-out holes where the tissue samples were taken. Each hole may be related to proteomics, genomics or other data that is then shown in a heat map. The digital image may thus show a removed section of FFPE tissue that was removed by the laser capture microdissect. The genomic data and the image of the corresponding tissue section plus the proteomic data and the corresponding tissue section are combined in a single viewer format that shows the relative abundance of certain genomic sequences and/or certain peptide or protein species, and further shows how they correlate with the image file. In some cases, before and after images may be taken of the tissue sample. The after images would show which sections were dissected (i.e. laser captured via microdissect).

Once the method of FIG. 3 has been performed for the first identified pixel 208, the method can be repeated for second and/or subsequent pixels. Each pixel need not be processed, and pixels need not be processed sequentially. Certain areas of the image may be identified by a user and rendered prior to other sections, or may be rendered to the exclusion of other sections. In other cases, random pixels may be accessed in the tissue sample digital image 213, and may be mapped with the various data sets 216 and 217, as described above. In this manner, a new representation may be created while using less processing resources than a method that maps every pixel in the image, or maps pixels in a strictly sequential manner. As each portion of the representation is rendered, that portion of the representation may be shown alone (e.g., FIG. 5), or in combination with the underlying tissue sample digital image 213 (e.g., FIGS. 6 and 7). In some cases, the renderable attributes may be shown in the same color (FIG. 6), and in other cases, the renderable attributes may be shown in different colors (FIG. 7).

In one specific embodiment, a system is provided for generating a representation of cell content of a tissue sample on a pixel-by-pixel basis. A pixel mapper 207 is configured to identify a first pixel 208 in a digital image 213 of a tissue sample. The identified first pixel 208 is mapped to a portion of the tissue sample. A data accessing module is provided which performs the following: accesses a first data set 216 representing a genomics analysis of nucleic acids that are separated from tissue proteins in the tissue sample, and accesses a second data set 217 representing a proteomic analysis of proteins that were separated from the nucleic acids of the tissue sample. The system also includes a processor 202 that is configured to map the accessed first and second data sets (216, 217) to a renderable attribute, so that the renderable attribute has a specified value based on the mapped data sets, and render at least a portion of a representation corresponding to the identified first pixel, according to the specified value of the renderable attribute. The representation includes an indication of how the separated nucleic acids and proteins are distributed within the tissue sample.

As noted above, the renderable attribute may be a heat value indicating the relative abundance of nucleic acids or proteins in the tissue sample. In other cases, the renderable attribute may be an indication of the presence or absence of other biological features or characteristics such as foreign bodies, abnormal cells, unusual cell groupings or other notable features. Indeed, it will be recognized that the renderable attributes may relate to any part of the image contents, regardless of the subject of the image. In cases where the newly generated representation is a heat map (e.g., FIGS. 6 and 7), the heat value at the first pixel and the heat values at other subsequently-mapped pixels are combined into a heat map overlay that represents where proteins or nucleic acids are found in each section of the tissue sample digital image. In some cases, a user may provide to the system one output file indicating output values of a genomics analysis, another output file indicating output values of a proteomics analysis, and an image of the tissue sample. The output files may be spreadsheet files from DNA and RNA analyses performed as described above. A graphic user interface (GUI) may be provided by the system that allows the user to easily input these analysis files and tissue sample image.

As shown in images 600 and 700 of FIGS. 6 and 7, respectively, the newly generated representation may be constructed pixel-by-pixel, and may include as a background, the digital image, and as a foreground one or more portions of proteomics quantitative measurement data and one or more portions of genomics quantitative measurement data. Data may be read from markers on the genomics side (e.g., the genomic analysis), from markers on the proteomic side, and image data from the digital image. This information is then combined and overlaced in a single image. Software executed by the system processor 201 may be configured to analyze the proteomics data, imaging data and genomics data to provide a big picture view of the content of the tissue sample 401. The proteomics data may be obtained from mass spectrometry, the genomic sequences may be obtained from lysing procedures (after the nucleic acid has been sequenced), and this output data may be used as the inputs to the software. The rendering module 210 then generates the rendered representation 211 that is provided to the display 212.

The system may be configured to perform all or part of a more specific method of extracting macromolecules from a biological sample and generating a representation thereof. This method includes providing a tissue sample having a plurality of cells containing nucleic acids and proteins, lysing the cells to produce a lysate containing at least a portion of the nucleic acids and proteins, alkylating, reducing, and enzymatically digesting the proteins in the lysate, separating the nucleic acids from the digested proteins, and performing nucleic acid analysis of the separated nucleic acids to produce a first data set, where the first data set represents a genomics analysis of the separated nucleic acids. The system also performs mass spectroscopic analysis of the separated digested proteins to produce a second data set, where the second data set represents a proteomic analysis of the separated digested proteins. The system further identifies a first pixel in a digital image of the tissue sample, which is mapped to a portion of the tissue sample and accesses the first data set and the second data set, mapping the accessed first and second data sets to a renderable attribute, such that the renderable attribute has a specified value based on the mapped data sets. The system also renders at least a portion of a representation corresponding to the identified first pixel, according to the specified value of the renderable attribute, where the representation includes an indication of how the nucleic acids and the proteins were distributed within the tissue sample. Such a method combines one or more parts of the methods described in FIGS. 1 and 3.

The renderable attribute may be a heat value, and the heat value may be represented as a specified color in a heat value image overlay. The heat value image overlay is then superimposed over the tissue sample digital image. The heat values may be color coded such that more abundant items are shown in a darker color and less abundant items are shown in a lighter color. In some cases, a user may specify which specific portions of the first and second data sets are to be used in the mapping for the heat map. Thus, the user may specify the data they are interested in, and cause the system to search through the proteomics and genomics data and pull out desired portions. This is then shown in conjunction with the digital image. The overlay representation shows a heat map with heat map values corresponding to the user-selected data sets. The overlays then show, on top of the image, a heat map that illustrates how much of each substance is found and where in the tissue section it is found. Thus, users have a great deal of control over which data sets are used in the image overlay, and even which portions of those data sets are used.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure. Moreover, unless a feature is described as requiring another feature in combination therewith, any feature herein may be combined with any other feature of a same or different embodiment disclosed herein. Furthermore, various well-known aspects of illustrative systems, processes, products, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

We claim:

1. A method for representing cell content of a tissue sample, the method comprising:
identifying a first pixel in a digital image of a tissue sample, wherein the identified first pixel is mapped to a portion of the tissue sample;
accessing a first data set representing a genomic analysis of one or more nucleic acids that are separated from one or more tissue proteins in the tissue sample;
accessing a second data set representing a proteomic analysis of one or more proteins that are separated from the one or more nucleic acids of the tissue sample;
mapping the accessed first and second data sets to a renderable attribute, such that the renderable attribute has a specified value based on the mapped data sets; and
rendering at least a portion of a representation corresponding to the identified first pixel, according to the specified value of the renderable attribute, the representation including an indication of how the separated nucleic acids and proteins are distributed within the tissue sample.

2. The method of claim 1, wherein the portion of the representation corresponding to the first pixel comprises an overlay superimposed over the first pixel of the tissue sample digital image.

3. The method of claim 2, wherein the overlay superimposed over the first pixel of the tissue sample digital image comprises a portion of a heat map representing the relative presence of separated nucleic acids and proteins.

4. The method of claim 1, wherein the first and second data sets comprise filtered lists of data, the data being filtered according to user-defined parameters.

5. The method of claim 1, further comprising performing a laser capture microdissect on a formalin-fixed paraffin-embedded (FFPE) tissue sample.

6. The method of claim 5, wherein the tissue sample comprises a laser capture microdissect FFPE tissue section.

7. The method of claim 5, wherein the digital image shows a portion of FFPE tissue that is remaining after the laser capture microdissect has been performed.

8. The method of claim 5, wherein the digital image shows a removed section of FFPE tissue that was removed by the laser capture microdissect.

9. The method of claim 1, further comprising repeating the method steps of claim 1 for at least a second pixel of the digital image.

10. The method of claim 9, wherein the method steps of claim 1 are repeated for each pixel in the digital image, thereby forming an overlay superimposed over the entire digital image.

11. A method of extracting macromolecules from a biological sample and generating a representation thereof, the method comprising:
providing a tissue sample having a plurality of cells containing nucleic acids and proteins;
lysing the cells to produce a lysate containing at least a portion of the nucleic acids and proteins;
alkylating, reducing, and enzymatically digesting the proteins in the lysate;
separating the nucleic acids from the digested proteins;
performing nucleic acid analysis of the separated nucleic acids to produce a first data set, the first data set representing a genomics analysis of the separated nucleic acids;
performing mass spectroscopic analysis of the separated digested proteins to produce a second data set, the second data set representing a proteomic analysis of the separated digested proteins;
identifying a first pixel in a digital image of the tissue sample, wherein the identified first pixel is mapped to a portion of the tissue sample;
accessing the first data set and the second data set;
mapping the accessed first and second data sets to a renderable attribute, such that the renderable attribute has a specified value based on the mapped data sets; and
rendering at least a portion of a representation corresponding to the identified first pixel, according to the specified value of the renderable attribute, the representation including an indication of how the nucleic acids and the proteins were distributed within the tissue sample.

12. The method of claim 11, wherein the renderable attribute comprises a heat value, and wherein the heat value is represented as a specified color in a heat value image overlay which is superimposed over the digital image.

13. The method of claim 11, wherein a user specifies one or more specific portions of the first and second data sets that are to be used in the mapping.

14. The method of claim 11, wherein the rendered representation includes an indication of differences between the digital image and the rendered representation.

15. A system for generating a representation of cell content of a tissue sample on a pixel-by-pixel basis, comprising:
a pixel mapper configured to identify a first pixel in a digital image of a tissue sample, wherein the identified first pixel is mapped to a portion of the tissue sample;
a data accessing module configured to perform the following:
access a first data set representing a genomics analysis of one or more nucleic acids that are separated from one or more tissue proteins in the tissue sample; and
access a second data set representing a proteomic analysis of one or more proteins that were separated from the one or more nucleic acids of the tissue sample; and
a processor configured to perform the following:
map the accessed first and second data sets to a renderable attribute, such that the renderable attribute has a specified value based on the mapped data sets; and
render at least a portion of a representation corresponding to the identified first pixel, according to the specified value of the renderable attribute, the representation including an indication of how the separated nucleic acids and proteins are distributed within the tissue sample.

16. The system of claim 15, wherein the renderable attribute comprises a heat value.

17. The system of claim 16, wherein the heat value indicates the relative presence or absence of separated tissue proteins and nucleic acids at the first pixel of the tissue sample digital image.

18. The system of claim 16, wherein the heat value at the first pixel and the heat values at one or more subsequent pixels are combined into a heat map overlay that represents where proteins or nucleic acids are found in each section of the tissue sample digital image.

19. The system of claim 15, wherein a user provides to the system a first output file indicating output values of a genomics analysis, one or more images of the tissue sample, and a second output file indicating output values of a proteomics analysis.

20. The system of claim 19, wherein the representation is constructed pixel-by-pixel, and includes as a background, the digital image, and as a foreground one or more portions of proteomics quantitative measurement data and one or more portions of genomics quantitative measurement data.

21. A system for generating a representation of cell content of a tissue sample on a pixel-by-pixel basis, comprising:
a nucleic acid analyzing element configured to produce a first data set representing a genomic analysis of one or more nucleic acids, the nucleic acid analyzing element comprising one or more apparatus selected from the group consisting of nucleic acid quantification devices, nucleic acid fragment detection devices, nucleic acid purity measurement devices, nucleic acid amplification devices, and nucleic acid sequencing devices;
a protein analyzing element configured to produce a second data set representing a proteomic analysis of one or more proteins, the protein analyzing element comprising one or more apparatus selected from the group consisting of protein purification devices and mass spectrometry devices;
a pixel mapper configured to identify a first pixel in a digital image of a tissue sample, the identified first pixel being mapped to a portion of the tissue sample;
a data accessing module configured to access the first and second data sets; and
a processor configured to perform the following:
map the accessed first and second data sets to a renderable attribute, such that the renderable attribute has a specified value based on the mapped data sets; and
render at least a portion of a representation corresponding to the identified first pixel, according to the specified value of the renderable attribute, the representation including an indication of how the one or more nucleic acids and the one or more proteins are distributed within the tissue sample.
